(19) United States
(12) Patent Application Publication
(10) Pub. No.: US 2025/0045926 A1
(43) Pub. Date: Feb. 6, 2025

(54) DETECTION OF ARTIFACTS IN SYNTHETIC IMAGES

(71) Applicant: BAYER AKTIENGESELLSCHAFT, Leverkusen (DE)

(72) Inventors: Matthias LENGA, Kiel (DE); Ivo Matteo BALTRUSCHAT, Hamburg (DE)

(21) Appl. No.: 18/776,651

(22) Filed: Jul. 18, 2024

(30) Foreign Application Priority Data
Jul. 25, 2023 (EP) 23187518.8

(51) Int. Cl. G06T 7/00; A61B 5/055; A61B 6/03; A61K 49/10; G06T 11/00; G06V 10/774; G06V 10/776

(52) U.S. Cl. CPC G06T 7/0016; A61B 5/055; A61B 6/032; A61K 49/105; A61K 49/108; G06T 11/001; G06V 10/774; G06V 10/776; G06T 2207/10024; G06T 2207/10081; G06T 2207/10088; G06T 2207/20081; G06T 2207/30004; G06T 2210/41

(57) ABSTRACT

The present disclosure relates to the technical field of generation of synthetic images, in particular synthetic medical images. The subjects of the present disclosure are a method, a computer system and a computer-readable storage medium comprising a computer program for detecting artifacts in synthetic images, in particular synthetic medical images.

### CROSS-REFERENCE TO RELATED APPLICATIONS

This application claims priority to European Patent Application No. 23187518.8, filed Jul. 25, 2023, the content of which is incorporated herein by reference in its entirety.

### FIELD OF THE DISCLOSURE

The present disclosure relates to the technical field of generation of synthetic images, in particular synthetic medical images. The subjects of the present disclosure are a method, a computer system and a computer-readable storage medium comprising a computer program for detecting artifacts in synthetic images, in particular synthetic medical images.

### BACKGROUND

Artificial intelligence is being increasingly adopted by medicine. Machine-learning models are not only being used to identify signs of disease in medical images of the human or animal body (see, for example, WO 2018/202541 A1, WO 2020/229152 A1), but are also being increasingly used to generate synthetic (artificial) medical images.

For example, WO 2021/052896 A1 and WO 2021/069338 A1 describe methods for generating an artificial medical image. The artificial medical image is generated with the aid of a trained machine-learning model on the basis of medical images showing the examination region of an examination object in a first period of time. The artificial medical image shows the examination region of the examination object in a second period of time that follows the first period of time. The method can be used, for example, to speed up radiological examinations; instead of measuring radiological images over a relatively long period of time, measurements are only made within one portion of the period of time and one or more radiological images are predicted for the remaining portion of the period of time with the aid of the trained model.

For example, WO 2019/074938 A1 and WO 2022/184297 A1 describe methods for generating an artificial radiological image showing an examination region of an examination object after the administration of a standard amount of a contrast agent, even though only a lower amount of contrast agent than the standard amount has been administered. The standard amount is the amount recommended by the manufacturer and/or distributor of the contrast agent and/or the amount approved by a regulatory authority and/or the amount specified in a package leaflet for the contrast agent. The methods described in WO 2019/074938 A1 and WO 2022/184297A can therefore be used to reduce the amount of contrast agent.

The medical images generated by the trained machine-learning models may contain errors (see for example: K. Schwarz et al.: On the Frequency Bias of Generative Models, doi.org/10.48550/arXiv.2111.02447).

Such errors can be problematic, since a doctor might make a diagnosis and/or initiate therapy on the basis of the artificial medical images. When a doctor reviews artificial medical images, he/she needs to know whether features in the artificial medical images are due to real features of the examination object or whether they are artifacts due to errors in prediction by the trained machine-learning model.

### SUMMARY

These and other problems are addressed by the subjects of the present disclosure.

The present disclosure provides in a first aspect a computer-implemented method for generating at least one confidence value for a synthetic image, comprising the steps of:
providing a trained machine-learning model,
   where the trained machine-learning model has been trained on the basis of training data,
   where the training data comprise for each reference object of a multiplicity of reference objects (i) at least one input reference image of a reference region of the reference object in a first state and (ii) a target reference image of the reference region of the reference object in a second state, where the at least one input reference image and the target reference image each comprise a multiplicity of image elements,
   where the machine-learning model is configured and has been trained to generate for each reference object on the basis of the at least one input reference image a synthetic reference image,
      where the synthetic reference image comprises a multiplicity of image elements, where each image element of the synthetic reference image respectively corresponds to an image element of the target reference image,
      where the machine-learning model has been trained to predict for each image element of the synthetic reference image a color value and an uncertainty value for the predicted color value,
      where the training comprises minimization of a loss function, where the loss function comprises (i) the predicted color value and/or a deviation of the predicted color value from a color value of the corresponding image element of the target reference image and (ii) the predicted uncertainty value as parameters,
receiving at least one input image of an examination region of an examination object, where the at least one input image represents the examination region of the examination object in the first state,
feeding the at least one input image to the trained machine-learning model,
receiving a synthetic image from the trained machine-learning model, where the synthetic image represents the examination region of the examination object in the second state,
receiving an uncertainty value for each image element of the synthetic image,
determining at least one confidence value on the basis of the received uncertainty values,
outputting the at least one confidence value.

The present disclosure further provides a computer-readable storage medium comprising a computer program which can be loaded into a working memory of a computer system, where it causes the computer system to execute the following steps:
providing a trained machine-learning model,
   where the trained machine-learning model has been trained on the basis of training data,
   where the training data comprise for each reference object of a multiplicity of reference objects (i) at least one input reference image of a reference region of the reference object in a first state and (ii) a target reference image of the reference region of the reference object in a second state, where the at least one input reference image and the target reference image each comprise a multiplicity of image elements,
   where the machine-learning model is configured and has been trained to generate for each reference object on the basis of the at least one input reference image a synthetic reference image,
      where the synthetic reference image comprises a multiplicity of image elements, where each image element of the synthetic reference image respectively corresponds to an image element of the target reference image,
      where the machine-learning model has been trained to predict for each image element of the synthetic reference image a color value and an uncertainty value for the predicted color value,
      where the training comprises minimization of a loss function, where the loss function comprises (i) the predicted color value and/or a deviation of the predicted color value from a color value of the corresponding image element of the target reference image and (ii) the predicted uncertainty value as parameters,
receiving at least one input image of an examination region of an examination object, where the at least one input image represents the examination region of the examination object in the first state,
feeding the at least one input image to the trained machine-learning model,
receiving a synthetic image from the trained machine-learning model, where the synthetic image represents the examination region of the examination object in the second state,
receiving an uncertainty value for each image element of the synthetic image,
determining at least one confidence value on the basis of the received uncertainty values,
outputting the at least one confidence value.

The present disclosure further provides a contrast agent for use in a radiological examination method, wherein the radiological examination method comprises:
providing a trained machine-learning model,
   where the trained machine-learning model has been trained on the basis of training data,
   where the training data comprise for each reference object of a multiplicity of reference objects (i) at least one radiological input reference image of a reference region of the reference object in a first state and (ii) a radiological target reference image of the reference region of the reference object in a second state,
   where the first state represents the reference region of the reference object in a first period of time before and/or after the administration of the contrast agent and the second state represents the reference region of the reference object in a second period of time after the administration of the contrast agent, and/or the first state represents the reference region of the reference object before and/or after the administration of a first amount of the contrast agent and the second state represents the reference region of the reference object after the administration of a second amount of the contrast agent,
   where the at least one radiological input reference image and the radiological target reference image each comprise a multiplicity of image elements,
   where the machine-learning model is configured and has been trained to generate for each reference object on the basis of the at least one radiological input reference image a synthetic radiological reference image,
      where the synthetic radiological reference image comprises a multiplicity of image elements, where each image element of the synthetic radiological reference image respectively corresponds to an image element of the radiological target reference image,
      where the machine-learning model has been trained to predict for each image element of the synthetic radiological reference image a color value and an uncertainty value for the predicted color value,
      where the training comprises minimization of a loss function, where the loss function comprises (i) the predicted color value and/or a deviation of the predicted color value from a color value of the corresponding image element of the radiological target reference image and (ii) the predicted uncertainty value as parameters,
receiving or generating at least one radiological input image of an examination region of an examination object, where the at least one radiological input image represents the examination region of the examination object in the first state,
feeding the at least one radiological input image to the trained machine-learning model,
receiving a synthetic radiological image from the trained machine-learning model, where the synthetic radiological image represents the examination region of the examination object in the second state,
receiving an uncertainty value for each image element of the synthetic radiological image,
determining at least one confidence value on the basis of the received uncertainty values,
outputting the at least one confidence value.

The present disclosure further provides for the use of a contrast agent in a radiological examination method, wherein the radiological examination method comprises:
providing a trained machine-learning model,
   where the trained machine-learning model has been trained on the basis of training data,
   where the training data comprise for each reference object of a multiplicity of reference objects (i) at least one radiological input reference image of a reference region of the reference object in a first state and (ii) a radiological target reference image of the reference region of the reference object in a second state,
   where the first state represents the reference region of the reference object in a first period of time before and/or after the administration of the contrast agent and the second state represents the reference region of the reference object in a second period of time after the administration of the contrast agent, and/or the first state represents the reference region of the reference object before and/or after the administration of a first amount of the contrast agent and the second state represents the reference region of the reference object after the administration of a second amount of the contrast agent,
   where the at least one radiological input reference image and the radiological target reference image each comprise a multiplicity of image elements,
   where the machine-learning model is configured and has been trained to generate for each reference object on the basis of the at least one radiological input reference image a synthetic radiological reference image,
      where the synthetic radiological reference image comprises a multiplicity of image elements, where each image element of the synthetic radiological reference image respectively corresponds to an image element of the radiological target reference image,
      where the machine-learning model has been trained to predict for each image element of the synthetic radiological reference image a color value and an uncertainty value for the predicted color value,
      where the training comprises minimization of a loss function, where the loss function comprises (i) the predicted color value and/or a deviation of the predicted color value from a color value of the corresponding image element of the radiological target reference image and (ii) the predicted uncertainty value as parameters,
receiving or generating at least one radiological input image of an examination region of an examination object, where the at least one radiological input image represents the examination region of the examination object in the first state,
feeding the at least one radiological input image to the trained machine-learning model,
receiving a synthetic radiological image from the trained machine-learning model, where the synthetic radiological image represents the examination region of the examination object in the second state,
receiving an uncertainty value for each image element of the synthetic radiological image,
determining at least one confidence value on the basis of the received uncertainty values,
outputting the at least one confidence value.

The present disclosure further provides a kit comprising a contrast agent and a computer program which can be loaded into a working memory of a computer system, where it causes the computer system to execute the following steps:
providing a trained machine-learning model,
   where the trained machine-learning model has been trained on the basis of training data,
   where the training data comprise for each reference object of a multiplicity of reference objects (i) at least one radiological input reference image of a reference region of the reference object in a first state and (ii) a radiological target reference image of the reference region of the reference object in a second state,
   where the first state represents the reference region of the reference object in a first period of time before and/or after the administration of the contrast agent and the second state represents the reference region of the reference object in a second period of time after the administration of the contrast agent, and/or the first state represents the reference region of the reference object before and/or after the administration of a first amount of the contrast agent and the second state represents the reference region of the reference object after the administration of a second amount of the contrast agent,
   where the at least one radiological input reference image and the radiological target reference image each comprise a multiplicity of image elements,
   where the machine-learning model is configured and has been trained to generate for each reference object on the basis of the at least one radiological input reference image a synthetic radiological reference image,
      where the synthetic radiological reference image comprises a multiplicity of image elements, where each image element of the synthetic radiological reference image respectively corresponds to an image element of the radiological target reference image,
      where the machine-learning model has been trained to predict for each image element of the synthetic radiological reference image a color value and an uncertainty value for the predicted color value,
      where the training comprises minimization of a loss function, where the loss function comprises (i) the predicted color value and/or a deviation of the predicted color value from a color value of the corresponding image element of the radiological target reference image and (ii) the predicted uncertainty value as parameters,
receiving or generating at least one radiological input image of an examination region of an examination object, where the at least one radiological input image represents the examination region of the examination object in the first state,
feeding the at least one radiological input image to the trained machine-learning model,
receiving a synthetic radiological image from the trained machine-learning model, where the synthetic radiological image represents the examination region of the examination object in the second state,
receiving an uncertainty value for each image element of the synthetic radiological image,
determining at least one confidence value on the basis of the received uncertainty values,
outputting the at least one confidence value.

Further subjects of the present disclosure are disclosed in the following description, the dependent claims and the drawings.

### BRIEF DESCRIPTION OF THE DRAWINGS

FIG. 1 shows by way of example and in schematic form a method for training the machine-learning model.

FIG. 2 shows by way of example and in schematic form a computer-implemented method for generating a synthetic image of an examination region of an examination object.

FIG. 3 shows one embodiment of the method for training the machine-learning model in the form of a flowchart.

FIG. 4 shows in schematic form a further embodiment of the computer-implemented method for training the machine-learning model.

FIG. 5 shows one embodiment of the method for generating at least one confidence value for a synthetic image.

FIG. 6 shows in schematic form a further embodiment of the computer-implemented method for generating a synthetic image of an examination region of an examination object.

FIG. 7 shows by way of example and in schematic form a computer system according to the present disclosure.

FIG. 8 shows by way of example and in schematic form a further embodiment of the computer system of the present disclosure.

### DETAILED DESCRIPTION

The disclosure will be more particularly elucidated below without distinguishing between the subjects of the present disclosure (method, computer system, computer-readable storage medium, use, contrast agent for use, kit). Rather, the following statements shall apply mutatis mutandis to all the subjects of the disclosure, irrespective of the context (method, computer system, computer-readable storage medium, use, contrast agent for use, kit) in which they are described.

Where steps are stated in an order in the present description or in the claims, this does not necessarily mean that the disclosure is limited to the order stated. Rather, it is conceivable that the steps can also be executed in a different order or else in parallel with one another, unless one step builds on another step, which absolutely requires that the step building on the previous step be executed subsequently (this will, however, become clear in the individual case).

In certain places the disclosure will be more particularly elucidated with reference to drawings. The drawings show specific embodiments having specific features and combinations of features, which are intended primarily for illustrative purposes; the disclosure is not to be understood as being limited to the features and combinations of features shown in the drawings. Furthermore, statements made in the description of the drawings in relation to features and combinations of features are intended to be generally applicable, that is to say transferable to other embodiments too and not limited to the embodiments shown.

The present disclosure describes means for judging the trustworthiness of a synthetic image of an examination region of an examination object.

The term “trustworthiness” is understood to mean that a person reviewing the synthetic image is able to trust that structures and/or morphologies and/or textures depicted in the synthetic image are attributable to real structures and/or real morphologies and/or real textures of the examination region of the examination subject and are not artifacts.

The term “synthetic” as used herein may refer to a synthetic image that is not the direct result of a measurement on a real examination object, but has been artificially generated (calculated). However, a synthetic image can be based on images taken of a real examination object, i.e. one or more images taken of a real examination object can be used to generate the synthetic image. Examples of synthetic images are described in the introduction and in the further description of the present disclosure. According to the present disclosure, a synthetic image is generated by a machine-learning model. The generation of a synthetic image with the aid of a machine-learning model is also referred to as “prediction” in this description. The terms “synthetic” and “predicted” are used synonymously in this disclosure. In other words, a synthetic image is an image generated (predicted) by a (trained) machine-learning model on the basis of input data, which can comprise one or more images generated by measurement.

The “examination object” is preferably a human or an animal, preferably a mammal, most preferably a human.

The “examination region” is part of the examination object, for example an organ of a human or animal such as the liver, brain, heart, kidney, lung, stomach, intestines, pancreas, thyroid gland, prostate, breast, or part of the aforementioned organs, or multiple organs, or another part of the examination object.

The examination region may also include multiple organs and/or parts of multiple organs.

In one embodiment, the examination region includes a liver or part of a liver or the examination region is a liver or part of a liver of a mammal, preferably a human.

In a further embodiment, the examination region includes a brain or part of a brain or the examination region is a brain or part of a brain of a mammal, preferably a human.

In a further embodiment, the examination region includes a heart or part of a heart or the examination region is a heart or part of a heart of a mammal, preferably a human.

In a further embodiment, the examination region includes a thorax or part of a thorax or the examination region is a thorax or part of a thorax of a mammal, preferably a human.

In a further embodiment, the examination region includes a stomach or part of a stomach or the examination region is a stomach or part of a stomach of a mammal, preferably a human.

In a further embodiment, the examination region includes a pancreas or part of a pancreas or the examination region is a pancreas or part of a pancreas of a mammal, preferably a human.

In a further embodiment, the examination region includes a kidney or part of a kidney or the examination region is a kidney or part of a kidney of a mammal, preferably a human.

In a further embodiment, the examination region includes one or both lungs or part of a lung of a mammal, preferably a human.

In a further embodiment, the examination region includes a breast or part of a breast or the examination region is a breast or part of a breast of a female mammal, preferably a female human.

In a further embodiment, the examination region includes a prostate or part of a prostate or the examination region is a prostate or part of a prostate of a male mammal, preferably a male human.

The examination region, also referred to as the field of view (FOV), is in particular a volume that is imaged in radiological images. The examination region is typically defined by a radiologist, for example on a localized image. It is also possible for the examination region to be alternatively or additionally defined in an automated manner, for example on the basis of a selected protocol.

The term “image” refers to a data structure constituting a spatial distribution of a physical signal. The spatial distribution can have any dimension, for example 2D, 3D, 4D or a higher dimension. The spatial distribution can have any form, for example it can form a grid, which can be irregular or regular, and thereby define pixels or voxels. The physical signal can be any signal, for example proton density, echogenicity, permeability, absorption capacity, relaxivity, information about rotating hydrogen nuclei in a magnetic field, color, grey level, depth, surface or volume occupancy.

The term “image” is preferably understood to mean a two-, three- or higher-dimensional visually capturable representation of the examination region of the examination object. Such an image is usually a digital image. The term “digital” as used herein may mean that the image can be processed by a machine, generally a computer system. “Processing” is understood to mean the known methods for electronic data processing (EDP).

A digital image can be processed, edited and reproduced and also converted into standardized data formats, for example JPEG (graphics format of the Joint Photographic Experts Group), PNG (Portable Network Graphics) or SVG (Scalable Vector Graphics), by means of computer systems and software.

Digital images can be visualized by means of suitable display devices, for example computer monitors, projectors and/or printers.

In a digital image, image contents are usually represented by whole numbers and stored. In most cases, the images are two- or three-dimensional images, which can be binary coded and optionally compressed. The digital images are usually raster graphics, in which the image information is stored in a uniform raster grid. Raster graphics consist of a raster arrangement of so-called picture elements (pixels) in the case of two-dimensional representations or volume elements (voxels) in the case of three-dimensional representations. In the case of four-dimensional representations, the term doxel (dynamic voxel) is commonly used for the image elements. In the case of higher-dimensional representations or in general, the term “n-xel” is sometimes also used, where n indicates the particular dimension. This disclosure uses generally the term image element. An image element can therefore be a picture element (pixel) in the case of a two-dimensional representation, a volume element (voxel) in the case of a three-dimensional representation, a dynamic voxel (doxel) in the case of the four-dimensional representation or a higher-dimensional image element in the case of a higher-dimensional representation.

Each image element in an image is assigned a color value. The color value indicates how (e.g. in what color) the image element is to be visually displayed (e.g. on a monitor).

The simplest case is a binary image, in which an image element is displayed either white or black. The color value “0” is usually “black” and the color value “1” “white”.

In the case of a grey scale image, each image element is assigned a grey level, which ranges from black to white over a defined number of shades of grey. Grey levels are also referred to as grey values. The number of shades can range, for example, from 0 to 255 (i.e. 256 grey levels/grey values), and here too, the value “0” is usually “black” and the highest grey value (value of 255 in this example) “white”.

In the case of a color image, the color coding used for an image element is defined, inter alia, in terms of the color space and the color depth. In the case of an image, the color of which is defined in terms of the so-called RGB color space (RGB stands for the primary colors red, green and blue), each picture element is assigned three color values, one color value for the color red, one color value for the color green and one color value for the color blue. The color of an image element arises through the superimposition (additive blending) of the three color values. The individual color value can be discretized, for example, into 256 distinguishable levels, which are called tonal values and usually range from 0 to 255. The tonal value “0” of each color channel is usually the darkest color nuance. If all three color channels have the tonal value 0, the corresponding image element appears black; if all three color channels have the tonal value 255, the corresponding image element appears white.

Irrespective of whether the image is a binary image, a grey scale image or a color image, the term “color value” is used in this disclosure to indicate the color (including the “colors” “black” and “white” and all shades of grey) in which an image element is to be displayed. A color value can thus be, for example, a tonal value of a color channel, a shade of grey, or “black” or “white”.

A color value in an image (especially a medical image) usually represents a strength of a physical signal (see above). It should be noted that the “color value” can also be a value for the physical signal itself.

There are a multiplicity of possible digital image formats and color codings. For simplification, it is assumed in this description that the present images are raster graphics having a specific number of image elements. However, this assumption ought not in any way be understood as limiting. It is clear to a person skilled in the art of image processing how the teaching of this description can be applied to image files which are present in other image formats and/or in which the color values are coded differently.

An “image” in the context of the present disclosure can also be one or more excerpts from a video sequence.

In a first step, at least one input image of an examination region of an examination object is received.

The term “receiving” encompasses both the retrieving of images and the accepting of images transmitted, for example, to the computer system of the present disclosure. The at least one input image can be received from a computed tomography scanner, from a magnetic resonance imaging scanner, from an ultrasound scanner, from a camera and/or from some other device for generating images. The at least one input image can be read from a data memory and/or transmitted from a separate computer system.

The expression “receiving at least one input image” as used herein may mean that one or more input images are received. It is thus possible to receive one input image; however, it is also possible to receive two or three or four or more than four input images.

The additional word “input” in the term “input image” indicates that the input image is intended to be fed to a (trained) machine-learning model as input data or at least as part of the input data.

Preferably, the at least one input image is a two-dimensional or three-dimensional representation of an examination region of an examination object.

In one embodiment of the present disclosure, the at least one input image is a medical image.

A “medical image” is a visual representation of an examination region of a human or animal that can be used for diagnostic and/or therapeutic purposes.

There is a multitude of techniques that can be used to generate medical images; examples of such techniques include radiography, computed tomography (CT), fluoroscopy, magnetic resonance imaging (MRI), ultrasound (sonography), endoscopy, elastography, tactile imaging, thermography, microscopy, positron emission tomography, optical coherence tomography (OCT), fundus photography and others.

Examples of medical images include CT images, X-ray images, MRI images, fluorescence angiography images, OCT images, histological images, ultrasound images, fundus images and/or others.

The at least one input image can be a CT image, MRI image, ultrasound image, OCT image and/or some other representation of an examination region of an examination object.

The at least one input image can also include representations of different modalities, for example one or more CT images and one or more MRI images.

Preferably, the at least one input image is the result of a radiological examination. In other words, the at least one input image is preferably a radiological image.

“Radiology” is the branch of medicine that is concerned with the use of electromagnetic rays and mechanical waves (including for instance ultrasound diagnostics) for diagnostic, therapeutic and/or scientific purposes. Besides X-rays, other ionizing radiation such as gamma radiation or electrons are also used. Imaging being a key application, other imaging methods such as sonography and magnetic resonance imaging (nuclear magnetic resonance imaging) are also counted as radiology, even though no ionizing radiation is used in these methods. The term “radiology” in the context of the present disclosure thus encompasses in particular the following examination methods: computed tomography, magnetic resonance imaging, sonography.

In one embodiment of the present disclosure, the radiological examination is a magnetic resonance imaging examination, i.e. the at least one input image comprises at least one MRI image.

In a further embodiment, the radiological examination is a computed tomography examination, i.e. the at least one input image comprises at least one CT image.

In a further embodiment, the radiological examination is an ultrasound examination, i.e. the at least one input image comprises at least one ultrasound image.

In radiological examinations, contrast agents are commonly used for contrast enhancement.

“Contrast agents” are substances or mixtures of substances that improve the depiction of structures and functions of the body in radiological examinations.

In computed tomography, iodine-containing solutions are normally used as contrast agents. In magnetic resonance imaging (MRI), superparamagnetic substances (for example iron oxide nanoparticles, superparamagnetic iron-platinum particles (SIPPs)) or paramagnetic substances (for example gadolinium chelates, manganese chelates, hafnium chelates) are normally used as contrast agents. In the case of sonography, liquids containing gas-filled microbubbles are normally administered intravenously. Examples of contrast agents can be found in the literature (see, for example, A. S. L. Jascinth et al.: Contrast Agents in computed tomography: A Review, Journal of Applied Dental and Medical Sciences, 2016, Vol. 2, Issue 2, 143-149; H. Lusic et al.: X-ray-Computed Tomography Contrast Agents, Chem. Rev. 2013, 113, 3, 1641-1666; radiology.wisc.edu/wp-content/uploads/2017/10/contrast-agents-tutorial.pdf, M. R. Nough et al.: Radiographic and magnetic resonances contrast agents: Essentials and tips for safe practices, World J Radiol. 2017 Sep. 28; 9(9): 339-349; L. C. Abonyi et al.: Intravascular Contrast Media in Radiography: Historical Development & Review of Risk Factors for Adverse Reactions, South American Journal of Clinical Research, 2016, Vol. 3, Issue 1, 1-10; ACR Manual on Contrast Media, 2020, ISBN: 978-1-55903-012-0; A. Ignee et al.: Ultrasound contrast agents, Endosc Ultrasound. 2016 November-December; 5(6): 355-362).

MRI contrast agents exert their effect in an MRI examination by altering the relaxation times of structures that take up contrast agents. A distinction can be made between two groups of substances: paramagnetic and superparamagnetic substances. Both groups of substances have unpaired electrons that induce a magnetic field around the individual atoms or molecules. Superparamagnetic contrast agents result in a predominant shortening of T2, whereas paramagnetic contrast agents mainly result in a shortening of T1. The effect of said contrast agents is indirect, since the contrast agent does not itself emit a signal, but instead merely influences the intensity of signals in its vicinity. An example of a superparamagnetic contrast agent is iron oxide nanoparticles (SPIO, superparamagnetic iron oxide). Examples of paramagnetic contrast agents are gadolinium chelates such as gadopentetate dimeglumine (trade name: Magnevist® and others), gadoteric acid (Dotarem©, Dotagita®, Cyclolux®), gadodiamide (Omniscan®), gadoteridol (ProHance®), gadobutrol (Gadovist®), gadopiclenol (Elucirem, Vueway) and gadoxetic acid (Primovist®/Eovist®).

In one embodiment, the radiological examination is an MRI examination in which an MRI contrast agent is used.

In a further embodiment, the radiological examination is a CT examination in which a CT contrast agent is used.

In a further embodiment, the radiological examination is a CT examination in which an MRI contrast agent is used.

In one embodiment of the present disclosure, the contrast agent is an agent that includes gadolinium(III) 2-[4,7,10-tris(carboxymethyl)-1,4,7,10-tetrazacyclododec-1-yl]acetic acid (also referred to as gadolinium-DOTA or gadoteric acid).

In a further embodiment, the contrast agent is an agent that includes gadolinium(III) ethoxybenzyldiethylenetriaminepentaacetic acid (Gd-EOB-DTPA); preferably, the contrast agent includes the disodium salt of gadolinium(III) ethoxybenzyldiethylenetriaminepentaacetic acid (also referred to as gadoxetic acid).

In one embodiment of the present disclosure, the contrast agent is an agent that includes gadolinium(III) 2-[3,9-bis[1-carboxylato-4-(2,3-dihydroxypropylamino)-4-oxobutyl]-3,6,9,15-tetrazabicyclo[9.3.1]pentadeca-1(15),11,13-trien-6-yl]-5-(2,3-dihydroxypropylamino)-5-oxopentanoate (also referred to as gadopiclenol) (see for example WO2007/042504 and WO2020/030618 and/or WO2022/013454).

In one embodiment of the present disclosure, the contrast agent is an agent that includes dihydrogen [(±)-4-carboxy-5,8,11-tris(carboxymethyl)-1-phenyl-2-oxa-5,8,11-triazatridecan-13-oato(5-)]gadolinate(2-) (also referred to as gadobenic acid).

In one embodiment of the present disclosure, the contrast agent is an agent that includes tetragadolinium [4,10-bis(carboxylatomethyl)-7-{3,6,12,15-tetraoxo-16-[4,7,10-tris-(carboxylatomethyl)-1,4,7,10-tetraazacyclododecan-1-yl]-9,9-bis({[({2-[4,7,10-tris-(carboxylatomethyl)-1,4,7,10-tetraazacyclododecan-1-yl]propanoyl}amino)acetyl]amino}methyl)-4,7,11,14-tetraazaheptadecan-2-yl}-1,4,7,10-tetraazacyclododecan-1-yl]acetate (also referred to as gadoquatrane) (see for example J. Lohrke et al.: Preclinical Profile of Gadoquatrane: A Novel Tetrameric, Macrocyclic High Relaxivity Gadolinium-Based Contrast Agent. Invest Radiol., 2022, 1, 57(10): 629-638; WO2016193190).

In one embodiment of the present disclosure, the contrast agent is an agent that includes a Gd³, complex of a compound of the formula (I)

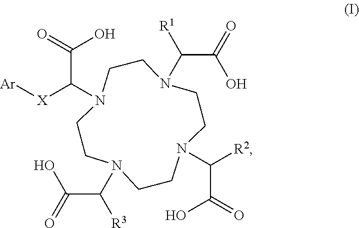

wherein:
Ar is a group selected from:

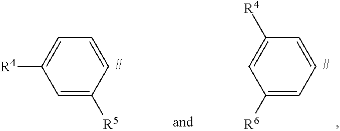

wherein _{#} is a linkage to X,
X is a group selected from:
CH₂, (CH₂)₂, (CH₂)₃, (CH₂)₄ and *—(CH₂)₂O—CH₂—^{#},
wherein * is a linkage to Ar and ^{#} is a linkage to an acetic acid residue,
R¹, R² and R³ are each independently a hydrogen atom or a group selected from C₁-C₃ alkyl, —CH₂OH, —(CH₂)₂OH and —CH₂OCH₃,
R⁴ is a group selected from C₂-C₄ alkoxy, (H₃C—CH₂)—O—(CH₂)₂—O—, (H₃C—CH₂)—O—(CH₂)₂—O—(CH₂)₂—O— and (H₃C—CH₂)—O—(CH₂)₂—O—(CH₂)₂—O—(CH₂)₂—O—,
R⁵ is a hydrogen atom,
and
R⁶ is a hydrogen atom,
or a stereoisomer, a tautomer, a hydrate, a solvate or a salt thereof, or a mixture thereof.

In one embodiment of the present disclosure, the contrast agent is an agent that includes a Gd³⁺ complex of a compound of the formula (II)

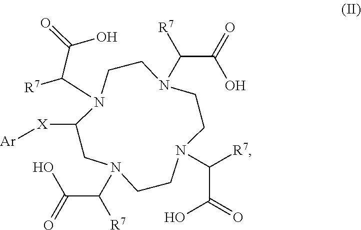

wherein:
Ar is a group selected from:

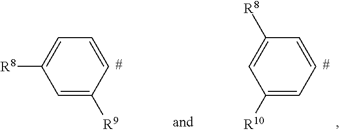

wherein _{#} is a linkage to X,
X is a group selected from:
CH₂, (CH₂)₂, (CH₂)₃, (CH₂)₄ and *—(CH₂)₂O—CH₂—^{#},
wherein * is a linkage to Ar and ^{#} is a linkage to an acetic acid residue,
R⁷ is a hydrogen atom or a group selected from C₁-C₃ alkyl, —CH₂OH, —(CH₂)₂OH and —CH₂OCH₃;
R⁸ is a group selected from:
C₂-C₄ alkoxy, (H₃C—CH₂O)—(CH₂)₂—O—, (H₃C—CH₂O)—(CH₂)₂—O—(CH₂)₂—O— and (H₃C—CH₂O)—(CH₂)₂—O—(CH₂)₂—O—(CH₂)₂—O—;
R⁹ and R¹⁰ are independently a hydrogen atom;
or a stereoisomer, a tautomer, a hydrate, a solvate or a salt thereof, or a mixture thereof.

The term “C₁-C₃ alkyl” denotes a linear or branched, saturated monovalent hydrocarbon group having 1, 2 or 3 carbon atoms, for example methyl, ethyl, n-propyl or isopropyl. The term “C₂-C₄ alkyl” denotes a linear or branched, saturated monovalent hydrocarbon group having 2, 3 or 4 carbon atoms.

The term “C₂-C₄ alkoxy” denotes a linear or branched, saturated monovalent group of the formula (C₂-C₄ alkyl)-O—, in which the term “C₂-C₄ alkyl” is as defined above, for example a methoxy, ethoxy, n-propoxy or isopropoxy group.

In one embodiment of the present disclosure, the contrast agent is an agent that includes gadolinium 2,2′,2″-(10-{1-carboxy-2-[2-(4-ethoxyphenyl)ethoxy]ethyl}-1,4,7,10-tetraazacyclododecane-1,4,7-triyl)triacetate (see for example WO2022/194777, example 1).

In one embodiment of the present disclosure, the contrast agent is an agent that includes gadolinium 2,2′,2″-{10-[1-carboxy-2-{4-[2-(2-ethoxyethoxy)ethoxy]phenyl}ethyl]-1,4,7,10-tetraazacyclododecane-1,4,7-triyl}triacetate (see for example WO2022/194777, example 2).

In one embodiment of the present disclosure, the contrast agent is an agent that includes gadolinium 2,2′,2″-{10-[(1R)-1-carboxy-2-{4-[2-(2-ethoxyethoxy)ethoxy]phenyl}ethyl]-1,4,7,10-tetraazacyclododecane-1,4,7-triyl}triacetate (see for example WO2022/194777, example 4).

In one embodiment of the present disclosure, the contrast agent is an agent that includes gadolinium (2S,2′S,2″S)-2,2′,2″-{10-[(1S)-1-carboxy-4-{4-[2-(2-ethoxyethoxy)ethoxy]phenyl}butyl]-1,4,7,10-tetraazacyclododecane-1,4,7-triyl}tris(3-hydroxypropanoate) (see for example WO2022/194777, example 15).

In one embodiment of the present disclosure, the contrast agent is an agent that includes gadolinium 2,2′,2″-{10-[(1S)-4-(4-butoxyphenyl)-1-carboxybutyl]-1,4,7,10-tetraazacyclododecane-1,4,7-triyl}triacetate (see for example WO2022/194777, example 31).

In one embodiment of the present disclosure, the contrast agent is an agent that includes gadolinium-2,2′,2″-{(2S)-10-(carboxymethyl)-2-[4-(2-ethoxyethoxy)benzyl]-1,4,7,10-tetraazacyclododecane-1,4,7-triyl}triacetate.

In one embodiment of the present disclosure, the contrast agent is an agent that includes gadolinium 2,2′,2″-[10-(carboxymethyl)-2-(4-ethoxybenzyl)-1,4,7,10-tetraazacyclododecane-1,4,7-triyl]triacetate.

In one embodiment of the present disclosure, the contrast agent is an agent that includes gadolinium(III) 5,8-bis(carboxylatomethyl)-2-[2-(methylamino)-2-oxoethyl]-10-oxo-2,5,8,11-tetraazadodecane-1-carboxylate hydrate (also referred to as gadodiamide).

In one embodiment of the present disclosure, the contrast agent is an agent that includes gadolinium(III) 2-[4-(2-hydroxypropyl)-7,10-bis(2-oxido-2-oxoethyl)-1,4,7,10-tetrazacyclododec-1-yl]acetate (also referred to as gadoteridol).

In one embodiment of the present disclosure, the contrast agent is an agent that includes gadolinium(III) 2,2′,2″-(10-((2R,3S)-1,3,4-trihydroxybutan-2-yl)-1,4,7,10-tetraazacyclododecane-1,4,7-triyl)triacetate (also referred to as gadobutrol or Gd-DO3A-butrol).

The at least one input image can also include representations of the examination region that were generated under different measurement conditions, for example a T1-weighted MRI image and/or a T2-weighted MRI image and/or a diffusion-weighted MRI image and/or some other MRI image and/or one or more dual-energy CT images and/or one or more spectral CT images.

The at least one input image can also include multiple radiological images that were generated after administration of different amounts of a contrast agent and/or after administration of different contrast agents, for example a native radiological image and/or a radiological image after administration of a first amount of a contrast agent and/or one or more radiological images after administration of a second contrast agent and/or a virtual non-contrast representation (VNC representation).

The at least one input image can also include multiple radiological images that were generated at different times before and/or after the administration of one or more contrast agents and/or that represent the examination region in different phases and/or states.

The at least one input image comprises a multiplicity of image elements. Each image element of the multiplicity of image elements represents a sub-region of the examination region of the examination object. The terms “multiplicity of image elements” and “plurality of image elements” as used herein may mean at least 1,000, or at least 10,000, or at least 100,000, or more than 100,000. It is conceivable that the at least one input image comprises one or more image elements that do not represent the examination region of the examination object, but some other region such as an adjoining and/or surrounding region.

The at least one input image represents the examination region of the examination object in a first state.

If more than one input image is received (e.g. two or three or four or more than four), the further input images received can represent the examination region of the examination object in the same state as the first input image or in one or more further states. A first input image can represent the examination region of the examination object in, for example, a first state and a second input image can represent the examination region of the examination object in, for example, a second input state, the second state differing from the first state.

On the basis of at least one input image, the (trained) machine-learning model generates a synthetic image. The synthetic image represents the examination region of the examination object in a second state. The first state and the second state are different states.

If more than one input image is received and a synthetic image is generated on the basis of the multiple input images received, then the synthetic image represents the examination region of the examination object in a different state than any of the input images received that are used as the basis for generation of the synthetic image.

It is thus possible to receive a first input image and a second input image. The first input image can represent the examination region of the examination object in a first state and the second input image can represent the examination region of the examination object in a second state. The first state and the second state can be the same or different; preferably, they are different. A synthetic image can be created on the basis of the first input image and the second input image. The synthetic image represents the examination region of the examination object in a third state; the third state differs from the first state and the second state.

It is also possible to receive a first input image, a second input image and a third input image. The first input image can represent the examination region of the examination object in a first state; the second input image can represent the examination region of the examination object in a second state; the third input image can represent the examination region of the examination object in a third state. The first state, the second state and the third state can be the same or different; preferably, they are different. A synthetic image can be generated on the basis of the first input image, the second input image and the third input image. The synthetic image represents the examination region of the examination object in a fourth state; the fourth state differs from the first state, the second state and the third state.

Generally, a number m of input images can be received (where m is an integer greater than zero); each input image received represents the examination region of the examination object in one of p states, where p is an integer which can assume the values from 1 to m. The m input images received can be used as the basis to generate a synthetic image representing the examination region of the examination object in a state differing from the p states.

The state can be an amount of contrast agent that had been administered to the examination object. The state can be a period of time that has passed since the administration of a contrast agent. The state can be a specific contrast agent that had been administered to the examination object. The state can also be a state without contrast agent. The state can be a modality (e.g. MRI image, CT image, ultrasound image) or a measurement protocol leading to a specific appearance of the examination region in a medical image generated by measurement.

The at least one input image can comprise, for example, a radiological image of the examination region before and/or after administration of a first amount of a contrast agent and the synthetic image can be a synthetic radiological image of the examination region after the administration of a second amount of a contrast agent, the second amount being preferably greater than the first amount.

The at least one input image can comprise, for example, a radiological image of the examination region without contrast agent and/or with a lower amount of contrast agent than the standard amount of the contrast agent and the synthetic image can be a synthetic radiological image of the examination region after the administration of the standard amount of contrast agent (as described, for example, in WO 2019/074938 A1 or WO 2022/184297 A1).

The “standard amount” is normally the amount recommended by the manufacturer and/or distributor of the contrast agent and/or the amount approved by a regulatory authority and/or the amount specified in a package leaflet for the contrast agent. For example, the standard amount of Primovist® is 0.025 mmol Gd-EOB-DTPA disodium/kg body weight.

The at least one input image can comprise, for example, one or more MRI images representing the examination region before and/or after administration of the first amount of an MRI contrast agent and the synthetic image can be a synthetic MRI image after the administration of the second amount of the MRI contrast agent.

The at least one input image can also comprise a CT image before and/or after the administration of a first amount of an MRI contrast agent and the synthetic image can be a synthetic CT image after the administration of a second amount of an MRI contrast agent, the second amount being preferably greater than the first amount and preferably greater than the standard amount of the MRI contrast agent for MRI examinations (as described, for example, in WO 2023/161041 A1).

The at least one input image can comprise, for example, one or more radiological images of the examination region in a first period of time before and/or after the administration of a contrast agent and the synthetic image can be a synthetic radiological image of the examination region in a second period of time after the administration of the contrast agent, the second period of time preferably chronologically following the first period of time (as described, for example, in WO 2021/052896 A1).

The at least one input image can comprise, for example, one or more radiological images of the liver or part of the liver of an examination object that represent the liver or the part of the liver in the native phase (i.e. without contrast agent) and/or in the arterial phase and/or in the portal venous phase and/or in the transitional phase after the administration of a hepatobiliary contrast agent and the synthetic image can be a synthetic radiological image representing the liver or the part of the liver in the hepatobiliary phase after the administration of the hepatobiliary contrast agent. The one or more radiological images can be one or more MRI images and/or one or more CT images. The hepatobiliary contrast agent can be a hepatobiliary MRI contrast agent and/or hepatobiliary CT contrast agent.

The stated phases are, for example, described in more detail in the following publications: J. Magn. Reson. Imaging, 2012, 35(3): 492-511, doi:10.1002/jmri.22833; Clujul Medical, 2015, Vol. 88 no. 4: 438-448, DOI: 10.15386/cjmed-414; Journal of Hepatology, 2019, Vol. 71: 534-542, dx.doi.org/10.1016/j.jhep.2019.05.005).

A hepatobiliary contrast agent is understood to mean a contrast agent which is specifically taken up by healthy liver cells, the hepatocytes. Examples of hepatobiliary contrast agents are contrast agents based on gadoxetic acid. They are, for example, described in U.S. Pat. No. 6,039,931A. They are commercially available under the trade names Primovist® or Eovist® for example. A further contrast agent having a lower uptake into the hepatocytes is gadobenate dimeglumine (Multihance®). Further hepatobiliary contrast agents are described inter alia in WO 2022/194777.

The at least one input image can comprise, for example, one or more MRI images of an examination region of an examination object and the synthetic image can be a synthetic CT image of the examination region of the examination object.

The at least one input image can comprise, for example, one or more CT images of an examination region of an examination object and the synthetic image can be a synthetic MRI image of the examination region of the examination object.

The at least one input image can comprise, for example, one or more radiological images (e.g. MRI and/or CT images) of an examination region of an examination object that have been generated using a first measurement protocol and the synthetic image can be a synthetic radiological image of the examination region of the examination object using a second measurement protocol, i.e. a synthetic image showing the examination region of the examination object as it would look if the second measurement protocol had been applied instead of the first measurement protocol.

The synthetic image is generated (predicted) by means of a trained machine-learning model.

A “machine learning model” can be understood as meaning a computer-implemented data processing architecture. Such a model is able to receive input data and to supply output data on the basis of said input data and model parameters. Such a model is able to learn a relationship between the input data and the output data through training. During training, the model parameters can be adjusted so as to supply a desired output for a particular input.

During the training of such a model, the model is presented with training data from which it can learn. The trained machine-learning model is the result of the training process. Besides input data, the training data include the correct output data (target data) that are to be generated by the model on the basis of the input data. During training, patterns that map the input data onto the target data are identified.

In the training process, the input data of the training data are input into the model, and the model generates output data. The output data are compared with the target data. Model parameters can be altered so as to reduce the deviations between the output data and the target data to a (defined) minimum.

The training data comprise for each reference object of a multiplicity of reference objects (i) at least one input reference image of a reference region of the reference object in the first state and (ii) a target reference image of the reference region of the reference object in the second state as target data.

The term “reference” is used in this description to distinguish the phase of training the machine-learning model from the phase of using the trained model for prediction (i.e. for generation of synthetic images). The term “reference” otherwise has no limitation on meaning. Statements made in this description concerning at least one input image apply analogously to each input reference image; statements made in this description concerning the examination object apply analogously to each reference object; statements made in this description concerning the examination region apply analogously to the reference region.

Each reference object is, like the examination object, normally a living being, preferably a mammal, most preferably a human. The “reference region” is a part of the reference object. The reference region normally (but not necessarily) corresponds to the examination region of the examination object. In other words, when the examination region is an organ or part of an organ (for example the liver or part of the liver) of the examination object, the reference region of each reference object is preferably the corresponding organ or corresponding part of the organ of the respective reference object. The at least one input reference image of the reference region of the reference object in the first state thus usually corresponds to the at least one input image of the examination region of the examination object in the first state—with the difference that the at least one input reference image comes from the reference object and the at least one input image comes from the examination object.

The additional word “input” in the term “input reference image” indicates that the input reference image is intended to be fed to a machine-learning model as input data or at least as part of the input data in the context of training the machine-learning model.

The additional word “target” in the term “target reference image” indicates that the target reference image is used as target data (ground truth) or at least as part of the target data in the context of training the machine-learning model.

The target reference image is normally not a synthetic image, but the result of a measurement, for example a radiological examination.

The at least one input reference image comprises a multiplicity of image elements; each image element represents a sub-region of the reference region; each image element is characterized by a color value. Furthermore, the target reference image also comprises a multiplicity of image elements; each image element represents a sub-region of the reference region; each image element is characterized by a color value.

For each reference object, the at least one input reference image is fed to the machine-learning model. The machine-learning model is configured to generate on the basis of the at least one input reference image (and any further input data) a synthetic reference image. The synthetic reference image should come as close as possible to the (normally measured) target reference image.

The synthetic reference image comprises a multiplicity of image elements; each image element represents a sub-region of the reference region; each image element is characterized by a color value.

Each image element of the synthetic reference image respectively corresponds to an image element of the target reference image. Corresponding image elements are those image elements representing the same reference region (or examination region); corresponding image elements normally have the same coordinates.

The machine-learning model is configured and is trained to predict for each image element of the synthetic reference image a color value. The predicted color value of each image element should come as close as possible to the color value of the corresponding image element of the (normally measured) target reference image.

Furthermore, the machine-learning model is configured and is trained to predict for each predicted color value an uncertainty value. The uncertainty value reflects the uncertainty of the predicted color value.

Training of the machine-learning model comprises minimization of a loss function.

The loss function comprises the color value predicted by the machine-learning model and/or a deviation of the predicted color value from the color value of the corresponding image element of the target reference image as parameters. The loss function further comprises the uncertainty value predicted by the machine-learning model as a further parameter.

The loss function [symbol] can have, for example, the following equation (1):

$$ℒ = \frac{1}{N} {\sum }_{i = 1}^{N} \frac{1}{2 {\hat{σ} ( {x}_{i} )}^{2}} ‖ {y}_{i} - {\hat{y}}_{i} {‖}^{2} + \frac{1}{2} log {\hat{σ} ( {x}_{i} )}^{2} \qquad ( 1 )$$

Here, ŷᵢ is the predicted color value of the image element i of the synthetic reference image and yᵢ is the color value of the corresponding image element i of the (normally measured) target reference image. N indicates the number of image elements of the synthetic reference image. The term \hat{σ}(xᵢ) is a predicted uncertainty value; it depends on the color values xᵢ of the corresponding image elements of the at least one input reference image.

The loss function [symbol] stated here by way of example is based on the loss function proposed by A. Kendall and Y. Gal (see: A. Kendall, Y. Gal: What Uncertainties Do We Need in Bayesian Deep Learning for Computer Vision?, Computer Vision and Pattern Recognition, 2017, arxiv.org/abs/1703.04977).

The loss function [symbol] comprises a term corresponding to the L2 loss function (Euclidean distance):

$${\sum }_{i = 1}^{N} ‖ {y}_{i} - {\hat{y}}_{i} {‖}^{2}$$

If during training the machine-learning model predicts color values that come close to (ideally match) the color values of the target data, then the Euclidean distance is small (if the color values match, it is equal to zero) and the loss calculated using the loss function [symbol] is likewise small.

If the predicted color values deviate from the target data, then the Euclidean distance is large.

The Euclidean distance is divided in the loss function [symbol] by the square of the uncertainty value \hat{σ}(xᵢ). As a result, the first term of the loss function [symbol] remains small even in the event of a large Euclidean distance of the predicted color values from the color values of the target data. In other words, if the predicted color values deviate from the color values of the target data, the loss function [symbol] can still be minimized if the uncertainty values are large. In other words, a high deviation in the prediction of color values leads to a high uncertainty value.

In order that the model parameters are not altered in such a way during training of the machine-learning model that the uncertainty values become larger and larger (in order to minimize the loss function), there is a second term for regularization:

$$\frac{1}{2} log {\hat{σ} ( {x}_{i} )}^{2}$$

The machine-learning model thus implicitly learns the uncertainty values from the loss function; the uncertainty values are not part of the target data; the uncertainty values reflect the uncertainty of the input data, also referred to as heteroscedastic aleatoric uncertainty.

The above-indicated equation (1) is only one example of a loss function; other equations are conceivable.

Present in an equation for a suitable loss function are not only the predicted color values and/or deviations of the predicted color values from the color values of the target data but also one or more uncertainty values as parameters. Such an uncertainty value serves as a complement to the deviations of the predicted color values from the color values of the target data. It ensures that an increase in the deviations of the predicted color values from the color values of the target data is somewhat countered so to be able to minimize the loss function, even in the case of deviations different from zero.

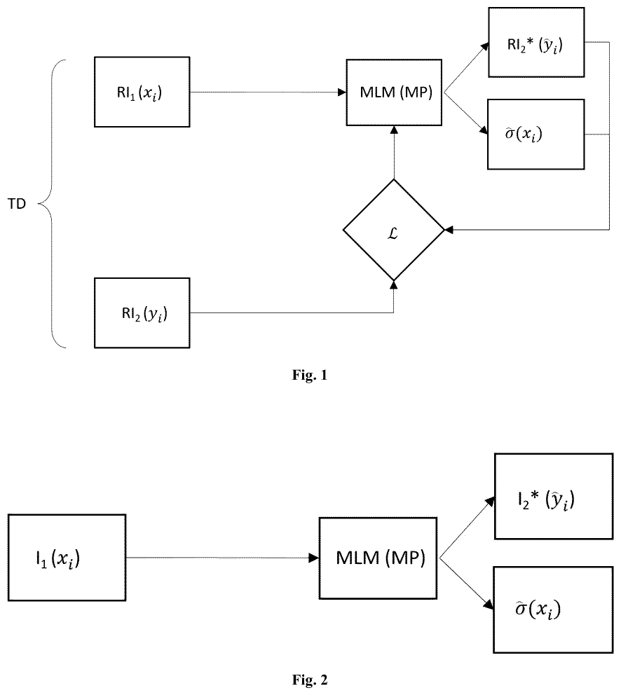

Training of the machine-learning model is shown by way of example and in schematic form in FIG. 1.

FIG. 1 shows by way of example and in schematic form a method for training the machine-learning model.

The machine-learning model MLM is trained using training data. The training data comprise for each reference object of a multiplicity of reference objects (i) at least one input reference image RI₁(xᵢ) of a reference region of the reference object in a first state as input data and (ii) a target reference image RI₂(yᵢ) of the reference region of the reference object in a second state as target data.

In the example shown in FIG. 1, one set of training data TD of one reference object is shown; normally, the training data comprise a multiplicity of these data sets for a multiplicity of reference objects.

The at least one input reference image RI₁(xᵢ) represents the reference region of the reference object in a first state; the target reference image RI₂(yᵢ) represents the reference region of the reference object in a second state. The first state and the second state are different states. For example, the state can represent an amount of contrast agent that is or has been introduced into the reference region. For example, the state can represent a specific contrast agent. For example, the state can represent a time before and/or after administration of a contrast agent. For example, the state can represent a modality and/or a measurement protocol. Further examples are mentioned in this disclosure.

The at least one input reference image RI₁(xᵢ) is used in the example shown in FIG. 1 as input data; it is fed to the machine-learning model MLM. The machine-learning model MLM is configured to generate on the basis of the at least one input reference image RI₁(xᵢ) and on the basis of model parameters MP a synthetic reference image RI₂*(ŷᵢ). The synthetic reference image RI₂*(ŷᵢ) is a synthetic (predicted) image. The synthetic reference image RI₂*(ŷᵢ) should come as close as possible to the target reference image RI₂(yᵢ). This means that the target reference image RI₂(yᵢ) acts in the example shown in FIG. 1 as target data (ground truth). The synthetic reference image RI₂*(ŷᵢ) should represent the reference region of the reference object in the second state.

The at least one input reference image RI₁(xᵢ) comprises a multiplicity of image elements (not explicitly shown in FIG. 1). Each image element i of the at least one input reference image RI₁(xᵢ) is characterized by a color value xᵢ. The target reference image RI₂(yᵢ) likewise comprises a multiplicity of image elements (not explicitly shown in FIG. 1). Each image element i of the target reference image RI₂(yᵢ) is characterized by a color value yᵢ.

The machine-learning model MLM is trained to predict for each image element i of the synthetic reference image RI₂*(ŷᵢ) a color value ŷᵢ. The prediction is made on the basis of the color values of the at least one input reference image RI₁(xᵢ).

Each predicted color value ŷᵢ of each image element of the synthetic reference image RI₂*(ŷᵢ) should come as close as possible (ideally correspond) to the respective color value yᵢ of the corresponding image element of the target reference image RI₂(yᵢ). In other words, deviations between the predicted color value ŷᵢ of an image element i of the synthetic reference image RI₂*(ŷᵢ) and the respective color value yᵢ of the corresponding image element i of the target reference image RI₂(yᵢ) should be as small as possible (ideally zero).

The machine-learning model MLM is furthermore trained to predict for each predicted color value ŷᵢ an uncertainty value \hat{σ}(xᵢ). The uncertainty value \hat{σ}(xᵢ) depends on the color values x of the image elements i of the at least one input reference image RI₁(xᵢ).

Training of the machine-learning model MLM comprises minimization of a loss function [symbol] . In other words, for each triple composed of a predicted color value ŷᵢ, the color value yᵢ and the predicted uncertainty value \hat{σ}(xᵢ), the loss function [symbol] can be used to calculate a loss; by modifying model parameters MP, the loss determined can be reduced; the goal can be that of modifying the model parameters in such a way that the loss determined by means of the loss function [symbol] assumes a minimum value for all triples and/or the loss determined cannot be reduced further by modifying the model parameters. The model parameters MP can be modified and the loss function [symbol] minimized in an optimization method. The loss function [symbol] can have the above-mentioned equation (1) or a different equation.

After training, the trained machine-learning model can be stored in a data memory and/or output and/or transmitted to another computer system. After training, the trained machine-learning model can be used to generate synthetic images.

FIG. 2 shows by way of example and in schematic form a computer-implemented method for generating a synthetic image of an examination region of an examination object.

The synthetic image is generated using a trained machine-learning model MLM^{t}. The trained machine-learning model MLM^{t} can have been trained as described in relation to FIG. 1.

The trained machine-learning model MLM^{t} is fed at least one input image I₁(xᵢ) of an examination region of an examination object. The at least one input image I₁(xᵢ) represents the examination region of the examination object in a first state. On the basis of the at least one input image I₁(xᵢ), the trained machine-learning model MLM^{t} generates a synthetic image I₂*(ŷᵢ). The synthetic image I₂*(ŷᵢ) represents the examination region of the examination object in a second state. The second state is different from the first state.

For example, the state can represent an amount of contrast agent that is or has been introduced into the examination region. For example, the state can represent a specific contrast agent. For example, the state can represent a time before and/or after administration of a contrast agent. For example, the state can represent a modality and/or a measurement protocol. Further examples are mentioned in this disclosure. The states during prediction normally correspond to the states during training of the machine-learning model.

The at least one input image I₁(xᵢ) comprises a multiplicity of image elements (not explicitly shown in FIG. 2); each image element i of the at least one input image I₁(xᵢ) represents a sub-region of the examination region; each image element i of the at least one input image I₁(xᵢ) is assigned a color value xᵢ.

The synthetic image I₂*(ŷᵢ) comprises a multiplicity of image elements (not explicitly shown in FIG. 2); each image element i of the synthetic image I₂*(ŷᵢ) represents a sub-region of the examination region.

The trained machine-learning model MLM^{t} is configured and has been trained to predict on the basis of the at least one input image I₁(xᵢ) a color value f for each image element i of the synthetic image I₂*(ŷᵢ). In other words, each image element i of the synthetic image I₂*(ŷᵢ) is assigned a predicted color value ŷᵢ.

The synthetic image I₂*(ŷᵢ) can be output (i.e. for example displayed on a monitor or printed by a printer) and/or stored in a data memory and/or transmitted to a separate computer system.

The trained machine-learning model MLM^{t} is furthermore configured and has furthermore been trained to predict for each predicted color value ŷᵢ an uncertainty value \hat{σ}(xᵢ).

The uncertainty values predicted for the predicted color values of the image elements of a synthetic image can be used as the basis to determine at least one confidence value.

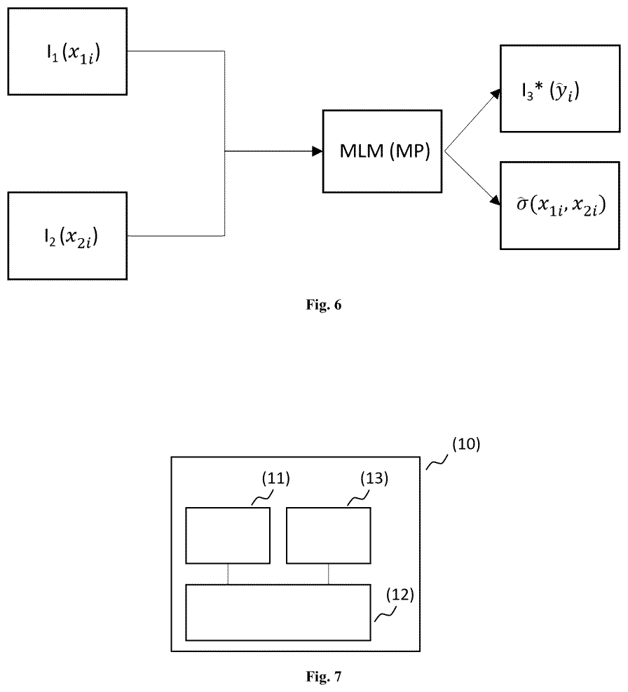

The at least one confidence value can be a value indicating the extent to which a synthetic image (e.g. synthetic image I₂*(ŷᵢ) in FIG. 2 or the synthetic image I₃*(ŷᵢ) in FIG. 6) can be trusted. The confidence value can positively correlate with the trustworthiness of the synthetic image, i.e. if the confidence value is low, trustworthiness is also low, and if the confidence value is high, trustworthiness is also high. However, it is also possible for the confidence value to negatively correlate with trustworthiness, i.e. if the confidence value is low, trustworthiness is high, and if the confidence value is high, trustworthiness is low.

If trustworthiness is low, then the synthetic image gives rise to a high degree of uncertainty; it is possible that the synthetic image has one or more artifacts; it is possible that structures and/or morphologies and/or textures in the synthetic image have no correspondence in reality, i.e. that structures and/or morphologies and/or textures in the synthetic image cannot be attributed to real structures and/or real morphologies and/or real textures in the examination region.

By contrast, a high confidence value indicates that the synthetic image has a low degree of uncertainty; features in the synthetic image have a correspondence in reality; the synthetic image can be trusted; a medical diagnosis can be made on the basis of the synthetic image and/or a medical therapy can be initiated on the basis of the synthetic image.

A confidence value positively correlating with trustworthiness can in principle be converted into a confidence value negatively correlating with trustworthiness, for example by forming the reciprocal (multiplicative inverse). Conversely, a confidence value negatively correlating with trustworthiness can correspondingly also be converted into a confidence value positively correlating with trustworthiness.

The uncertainty values predicted by the trained machine-learning model indicate for each individual image element the uncertainty of the respective predicted color value. Accordingly, these uncertainty values can be used directly as confidence values. They negatively correlate with trustworthiness; they positively correlate with uncertainty.

The at least one confidence value can also be a value derived from the uncertainty values.

Assuming that the predicted color values obey a Gaussian probability distribution, the predicted color value ŷᵢ in equation (1) corresponds to the mean, the uncertainty value \hat{σ}(xᵢ) to the standard deviation, and the square \hat{σ}(xᵢ)² of the uncertainty value to the variance of the Gaussian distribution function. Instead of the predicted uncertainty value \hat{σ}(xᵢ), the square \hat{σ}(xᵢ)² or some other derived variable can thus also be used as the at least one confidence value.

It is also conceivable that the machine-learning model is configured and is trained to predict a confidence value (instead of the uncertainty value or in addition to the uncertainty value). In such a case, the confidence value (or a variable derived therefrom) is a (further) parameter of the loss function.

It is thus possible to determine for each individual image element of the synthetic image a confidence value indicating the extent to which the color value of the image element can be trusted.

If there is more than one color value (e.g. three color values, as in the case of images, the color values of which are specified according to the RGB color model), an uncertainty value can be predicted for each color channel and a confidence value can be determined for each color channel.

However, it is also possible to combine the uncertainty values of the color channels into a single value and to determine a confidence value on the basis of the combined value; it is possible to determine a confidence value on the basis of the maximum uncertainty value of the color channels; it is possible to determine a confidence value on the basis of a mean (e.g. arithmetic mean, geometric mean, root mean square or some other mean) of the uncertainty values of the color channels; it is possible to determine a confidence value on the basis of the length of the vector specified by the uncertainty values of the color channels in a three-dimensional space (or a higher-dimensional space when using more than three color channels); other possibilities are conceivable.

It is possible to use different methods for calculating a confidence value for different sub-regions of the examination object. For example, it is possible that image elements representing a specific tissue and/or organ and/or lesion use a different method for calculating a confidence value than image elements representing another tissue and/or another organ and/or another sub-region. Sub-regions for which there are different calculation rules for confidence values can be identified, for example, by means of segmentation.

The term “segmentation” refers to the process of dividing an image into multiple segments, which are also referred to as image segments, image regions or image objects. Segmentation is generally used to locate objects and boundaries (lines, curves, etc.) in images. In a segmented image, the objects located can be separated from the background, visually highlighted (e.g. in color), measured, counted and/or quantified in some other way. In segmentation, each image element of an image is assigned a label (e.g. a number), and so image elements having the same label have certain features in common, for example represent the same tissue (e.g. bone tissue or adipose tissue or healthy tissue or diseased tissue (e.g. tumour tissue) or muscle tissue and/or the like) and/or the same organ. For image elements having a specific label, a specific calculation rule can then be used to calculate a confidence value; for image elements having a different (specific) label, a different (specific) calculation rule can be used to calculate a confidence value.

The confidence values determined for image elements can be output (e.g. displayed on a monitor or printed on a printer), stored in a data memory and/or transmitted to a separate computer system, for example via a network.

The confidence values determined for image elements can also be displayed pictorially.

In addition to the synthetic image generated on the basis of the at least one input image, a further representation of the examination region can thus be output (e.g. displayed on a monitor), indicating the trustworthiness for each image element. Such a representation is also referred to as confidence representation in this description. The confidence representation preferably has the same dimensions and size as the synthetic image; each image element of the synthetic image is preferably assigned an image element in the confidence representation.

Such a confidence representation can be used by a user (e.g. a doctor) to identify for each individual image element the extent to which the predicted color value of the image element can be trusted. It is possible to completely or partly superimpose the confidence representation on the synthetic image. It is possible to configure the superimposition in such a way that it can be faded in and out by the user. The user can display the synthetic image layer by layer, for example, as is customary for computed tomography representations, magnetic resonance imaging representations, and other three- or higher-dimensional representations. For each layer, the user can fade in the corresponding layer of the confidence representation in order to check whether the predicted color values of the image elements in the layer showing structures, morphologies, and/or textures are trustworthy or uncertain. This allows the user to identify the level of the risk of the structures, morphologies and/or textures being real properties of the examination region or artifacts.

For example, image elements having low trustworthiness (having a high degree of uncertainty) can be displayed brightly and/or with a signal color (e.g. red or orange or yellow), whereas image elements having high trustworthiness (having a low degree of uncertainty) can be displayed darkly or with a more inconspicuous or calming color (e.g. green or blue). It is also possible, in the case of a superimposition, to display those image elements for which the confidence value exceeds or falls short of a predefined limit value. If the confidence value correlates positively with trustworthiness, what can be displayed for example are those image elements of the confidence representation, the confidence value of which is below a predefined limit value; in such a case, a user (e.g. a doctor) is explicitly informed of those image elements that they should better not trust.

It is also possible to determine confidence values for sub-regions of the synthetic image (e.g. for layers within a three-dimensional or higher-dimensional synthetic image) and/or for the entire synthetic image. Determination of such confidence values for sub-regions or entire images can be done on the basis of the confidence values of those image elements of which they are composed. For example, a confidence value of a layer can be determined by taking into account all the confidence values of those image elements that lie in said layer. However, it is also possible to also take into account adjacent image elements (e.g. image elements of the layer above and/or below the layer under consideration). A confidence value for a sub-region or the entire region can be determined, for example, by forming a mean (e.g. arithmetic mean, geometric mean, root mean square or some other mean). It is also possible to determine the maximum value (e.g. in the case of a confidence value correlating negatively with trustworthiness) or the minimum value (e.g. in the case of a confidence value correlating negatively with trustworthiness) of the confidence values of the image elements of a sub-region or the entire region and to use it as the confidence value of the sub-region or the entire region. Further ways of determining a confidence value for a sub-region or the entire region on the basis of the confidence values of individual image elements are conceivable.

Such a confidence value for a sub-region or the entire region can be likewise output (e.g. displayed on a monitor or output on a printer), stored in a data memory and/or transmitted to a separate computer system. It can also be displayed pictorially (e.g. in color), as described for the individual confidence values.

If a confidence value for a sub-region or the entire region that correlates positively with trustworthiness is lower than a predefined limit value, then it is possible that the corresponding sub-region or entire region should not be trusted. It is possible that such a sub-region or the corresponding entire region is not output at all (e.g. not displayed at all), or that it is displayed with a warning indicating that a user should be careful when interpreting the displayed data owing to the uncertainty of the displayed data.

It is also possible that the user of the computer system/computer program of the present disclosure is given the option, via a user interface, of navigating in the synthetic image to sub-regions having low trustworthiness. For example, the user can be shown the sub-regions having the lowest trustworthiness in a list (e.g. in the form of a list having a number q of sub-regions having the lowest confidence value correlating positively with trustworthiness, where q is a positive integer). By clicking on a list entry, the user can be shown the corresponding sub-region in the form of a synthetic image, a confidence representation and/or an input image and/or a detail thereof.

The machine-learning model can for example be an artificial neural network or comprise such a network.

An artificial neural network comprises at least three layers of processing elements: a first layer with input neurons (nodes), an Nth layer with at least one output neuron (nodes) and N−2 inner layers, where N is a natural number and greater than 2.

The input neurons serve to receive the input data, in particular to receive the at least one input image. For example, there can be at least one input neuron for each image element of the at least one input image. Additional input neurons for additional input data can be present (e.g. information about the examination region, information about the examination object, information about the conditions when the at least one input image was generated, information about the first state and/or second state, and/or information about the time at which or period of time in which the at least one input image was generated).

The output neurons can serve to output a synthetic image.

The processing elements of the layers between the input neurons and the output neurons are connected to one another in a predetermined pattern with predetermined connection weights.

Preferably, the artificial neural network is a so-called convolutional neural network (CNN) or comprises such a network.

A CNN normally consists essentially of an alternately repeating array of filters (convolutional layer) and aggregation layers (pooling layer) terminating in one or more layers of fully connected neurons (dense/fully connected layer).

For example, the machine-learning model can have an architecture such as U-Net (see for example O. Ronneberger et al.: U-Net: Convolutional Networks for Biomedical Image Segmentation, arXiv:1505.04597v1). The machine-learning model can have an architecture as described in: V. P. Sudarshan et al.: Towards lower-dose PET using physics-based uncertainty-aware multimodal learning with robustness to out-of-distribution data, Medical Image Analysis 73 (2021) 102187.

The training of the neural network can, for example, be carried out by means of a backpropagation method. The aim here in respect of the network is maximum reliability of mapping of the at least one input reference image onto the target reference image. The quality of prediction is described by a loss function. The goal is to minimize the loss function. In the case of the backpropagation method, an artificial neural network is taught by the alteration of the connection weights.

In the trained state, the connection weights between the processing elements contain information regarding the relationships between a multiplicity of input reference images and the corresponding target reference images, which can be used for predictive purposes.

A cross-validation method can be used in order to divide the data into training and validation data sets. The training data set is used in the backpropagation training of network weights. The validation data set is used in order to check the accuracy of prediction with which the trained network can be applied to unknown data.

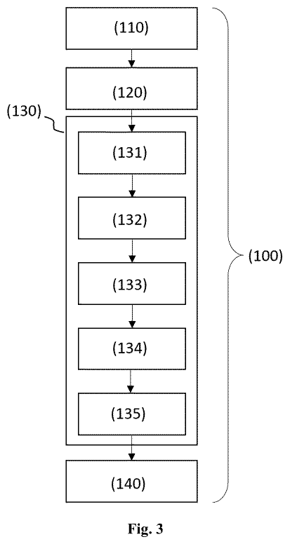

FIG. 3 shows one embodiment of the method for training the machine-learning model in the form of a flowchart.

The computer-implemented training method (100) comprises the steps of:
(110) receiving training data,
   where the training data comprise for each reference object of a multiplicity of reference objects (i) at least one input reference image of a reference region of the reference object in a first state and (ii) a target reference image of the reference region of the reference object in a second state,
   where the second state is different from the first state,
   where the at least one input reference image comprises a multiplicity of image elements, where each image element of the at least one input reference image represents a sub-region of the reference region, where each image element of the at least one input reference image is characterized by a color value,
   where the target reference image comprises a multiplicity of image elements, where each image element of the target reference image represents a sub-region of the reference region, where each image element of the target reference image is characterized by a color value,
(120) providing a machine-learning model,
   where the machine-learning model is configured to generate on the basis of the at least one input reference image of the reference region of a reference object and model parameters a synthetic reference image of the reference region of the reference object, where the synthetic reference image comprises a multiplicity of image elements, where each image element of the synthetic reference image corresponds to an image element of the target reference image, where each image element of the synthetic reference image is assigned a predicted color value, where the machine-learning model is configured to predict for each predicted color value an uncertainty value,
(130) training the machine-learning model, where the training for each reference object of the multiplicity of reference objects comprises:
   (131) inputting the at least one input reference image into the machine-learning model,
   (132) receiving a synthetic reference image from the machine-learning model,
   (133) receiving an uncertainty value for each predicted color value of the predicted second reference image,
   (134) calculating a loss by means of a loss function, where the loss function comprises (i) the predicted color value and/or a deviation between the predicted color value and a color value of the corresponding image element of the target reference image and (ii) the predicted uncertainty value as parameters,
   (135) reducing the loss by modification of model parameters,
(140) outputting and/or storing the trained machine-learning model and/or transmitting the trained machine-learning model to a separate computer system and/or using the trained machine-learning model to generate a synthetic image and/or to generate at least one confidence value for a synthetic image.

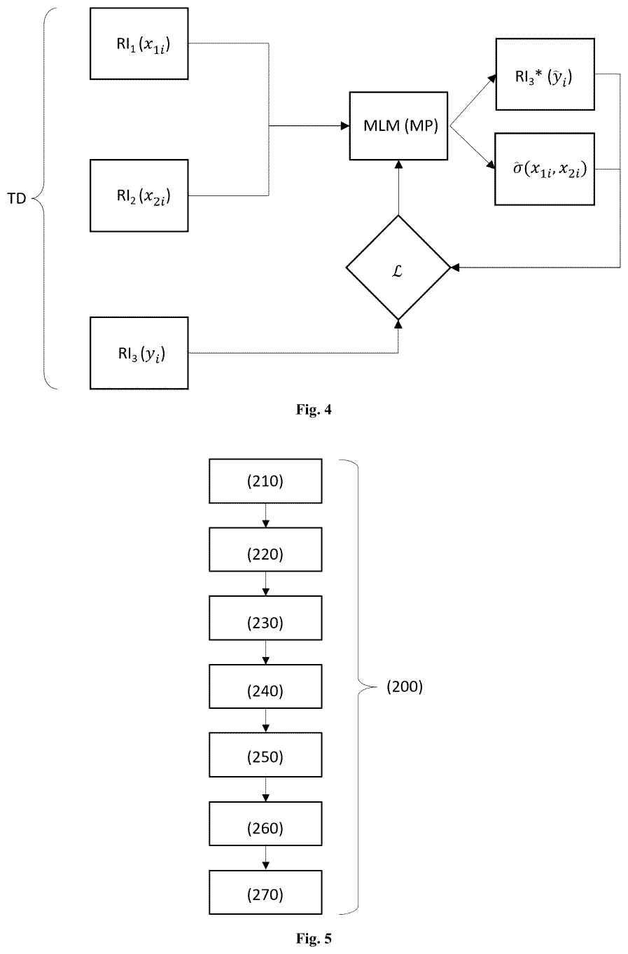

FIG. 4 shows in schematic form a further embodiment of the computer-implemented method for training the machine-learning model.

The machine-learning model MLM is trained using training data. The training data comprise for each reference object of a multiplicity of reference objects (i) at least one input reference image of a reference region of the reference object in a first state and (ii) a target reference image of the reference region of the reference object in a second state. The terms “multiplicity of reference objects” and “plurality of reference objects” as used herein may mean more than 10 and even more than 100 reference objects.

In the example shown in FIG. 4, one set of training data TD of one reference object is shown; normally, the training data comprise a multiplicity of these data sets for a multiplicity of reference objects. In the example shown in FIG. 4, the training data TD comprise a first input reference image RI₁(x₁ᵢ) and a second input reference image RI₂(x₂ᵢ) as input data and a target reference image RI₃(yᵢ) as target data.

The first input reference image RI₁(x₁ᵢ) represents the reference region of the reference object in a first state; the second input reference image RI₂(x₂ᵢ) represents the reference region of the reference object in a second state; the target reference image RI₃(yᵢ) represents the reference region of the reference object in a third state. The first state and the second state can be the same or different; preferably, the states are different. The third state is different from the first state and the second state. For example, the state can represent an amount of contrast agent that is or has been introduced into the reference region. For example, the state can represent a time before and/or after administration of a contrast agent.

For example, the first input reference image RI₁(x₁ᵢ) can represent the reference region without administration or after administration of a first amount of a contrast agent, the second input reference image RI₂(x₂ᵢ) can represent the reference region after administration of a second amount of the contrast agent, and the target reference image RI₃(yᵢ) can represent the reference region after administration of a third amount of the contrast agent. The first amount can be less than the second amount and the second amount can be less than the third amount (see for example WO 2019/074938 A1, WO 2022/184297 A1).

For example, the first input reference image RI₁(x₁ᵢ) can represent the reference region before administration or in a first period of time after administration of a contrast agent, the second input reference image RI₂(x₂ᵢ) can represent the reference region in a second period of time after administration of the contrast agent, and the target reference image RI₃(yᵢ) can represent the reference region in a third period of time after administration of the contrast agent (see for example WO 2021/052896 A1, WO 2021/069338 A1). The second period of time can follow the first period of time and the third period of time can follow the second period of time.

Further examples are mentioned in this disclosure.

The first input reference image RI₁(x₁ᵢ) and the second input reference image RI₂(x₂ᵢ) serve as input data in the example shown in FIG. 4; they are fed to the machine-learning model MLM. The machine-learning model MLM is configured to generate on the basis of the first input reference image RI₁(x₁ᵢ) and the second input reference image RI₂(x₂ᵢ) and on the basis of model parameters MP a synthetic reference image RI₃*(ŷᵢ). The synthetic reference image RI₃*(ŷᵢ) should come as close as possible to the target reference image RI₃(yᵢ). This means that the target reference image RI₃(yᵢ) acts in the example shown in FIG. 4 as target data (ground truth). The synthetic reference image RI₃*(ŷᵢ) should represent the reference region of the reference object in the third state.

The first input reference image RI₁(x₁ᵢ) comprises a multiplicity of image elements (not explicitly shown in FIG. 4). Each image element i of the first input reference image RI₁(x₁ᵢ) is characterized by a color value x₁ᵢ. The second input reference image RI₂(x₂ᵢ) likewise comprises a multiplicity of image elements (not explicitly shown in FIG. 4). Each image element i of the second input reference image RI₂(x₂ᵢ) is characterized by a color value x₂ᵢ. The target reference image RI₃(yᵢ) comprises a multiplicity of image elements (not explicitly shown in FIG. 4). Each image element i of the target reference image RI₃(yᵢ) is characterized by a color value yᵢ.

The machine-learning model MLM is trained to predict for each image element i of the synthetic reference image RI₃*(ŷᵢ) a color value ŷᵢ. The prediction is made on the basis of the color values x₁ᵢ of the first input reference image RI₁(x₁ᵢ) and the color values x₂ᵢ of the second input reference image RI₂(x₂ᵢ).

Each predicted color value ŷᵢ of each image element i of the synthetic reference image RI₃*(ŷᵢ) should come as close as possible (ideally correspond) to the respective color value yᵢ of the corresponding image element i of the target reference image RI₃(yᵢ). In other words, deviations between the predicted color value ŷᵢ and the respective color value yᵢ of the corresponding image element of the target reference image RI₃(yᵢ) should be as small as possible (ideally zero).

The machine-learning model MLM is furthermore trained to predict for each predicted color value ŷᵢ an uncertainty value \hat{σ}(x₁ᵢ, x₂ᵢ). The uncertainty value \hat{σ}(x₁ᵢ, x₂ᵢ) depends on the color values x₁ᵢ and x₂ᵢ of the image elements of the first input reference image RI₁(x₁ᵢ) and the second input reference image RI₂(x₂ᵢ).

Training of the machine-learning model MLM comprises minimization of a loss function [symbol] . In other words, for each triple composed of a predicted color value ŷᵢ, the color value yᵢ and the predicted uncertainty value \hat{σ}(x₁ᵢ, x₂ᵢ), the loss function [symbol] can be used to calculate a loss; by modifying model parameters MP, the loss determined can be reduced; the goal can be that of modifying the model parameters in such a way that the loss determined by means of the loss function [symbol] assumes a minimum value for all triples and/or the loss determined cannot be reduced further by modifying the model parameters. The model parameters MP can be modified and the loss function [symbol] minimized in an optimization method. The loss function [symbol] can have a form analogous to the above-mentioned equation (1) or be a different equation.

After training, the trained machine-learning model can be stored in a data memory and/or output and/or transmitted to another computer system. After training, the trained machine-learning model can be used to generate synthetic images.

The computer-implemented training method shown in FIG. 4 therefore comprises the following steps:
receiving training data,
   where the training data comprise for each reference object of a multiplicity of reference objects (i) a first input reference image of a reference region of the reference object in a first state and a second input reference image of the reference region of the reference object in a second state and (ii) a target reference image of the reference region of the reference object in a third state,
   where the second state is preferably different from the first state,
   where the third state is different from the first state and from the second state,
   where the first input reference image comprises a multiplicity of image elements, where each image element of the first input reference image represents a sub-region of the reference region, where each image element of the first input reference image is characterized by a color value,
   where the second input reference image comprises a multiplicity of image elements, where each image element of the second input reference image represents a sub-region of the reference region, where each image element of the second input reference image is characterized by a color value,
   where the target reference image comprises a multiplicity of image elements, where each image element of the target reference image represents a sub-region of the reference region, where each image element of the target reference image is characterized by a color value,
providing a machine-learning model,
   where the machine-learning model is configured to generate on the basis of a first input reference image of the reference region of a reference object, a second input reference image of the reference region of the reference object and model parameters a synthetic reference image of the reference region of the reference object, where the synthetic reference image comprises a multiplicity of image elements, where each image element of the synthetic reference image corresponds to an image element of the target reference image, where each image element of the synthetic reference image is assigned a predicted color value, where the machine-learning model is configured to predict for each predicted color value an uncertainty value,
training the machine-learning model, where the training for each reference object of the multiplicity of reference objects comprises:
   inputting the first input reference image and the second input reference image into the machine-learning model,
   receiving a synthetic reference image from the machine-learning model,
   receiving an uncertainty value for each predicted color value of the predicted third reference image,
   calculating a loss by means of a loss function, where the loss function comprises (i) the predicted color value and/or a deviation between the predicted color value and a color value of the corresponding image element of the target reference image and (ii) the predicted uncertainty value as parameters,
   reducing the loss by modification of model parameters,
outputting and/or storing the trained machine-learning model and/or transmitting the trained machine-learning model to a separate computer system and/or using the trained machine-learning model to generate a synthetic image and/or to generate at least one confidence value for a synthetic image.

FIG. 5 shows one embodiment of the method for generating at least one confidence value for a synthetic image in the form of a flow chart.

The computer-implemented method (200) comprises the steps of:
(210) providing a trained machine-learning model,
   where the trained machine-learning model has been trained on the basis of training data,
   where the training data comprise for each reference object of a multiplicity of reference objects (i) at least one input reference image of a reference region of the reference object in a first state and (ii) a target reference image of the reference region of the reference object in a second state, where the at least one input reference image and the target reference image each comprise a multiplicity of image elements,
   where the machine-learning model is configured and has been trained to generate for each reference object on the basis of the at least one input reference image a synthetic reference image,
      where the synthetic reference image comprises a multiplicity of image elements, where each image element of the synthetic reference image respectively corresponds to an image element of the target reference image,
      where the machine-learning model has been trained to predict for each image element of the synthetic reference image a color value and an uncertainty value for the predicted color value,
      where the training comprises minimization of a loss function, where the loss function comprises (i) the predicted color value and/or a deviation of the predicted color value from a color value of the corresponding image element of the target reference image and (ii) the predicted uncertainty value as parameters,
(220) receiving at least one input image of an examination region of an examination object, where the at least one input image represents the examination region of the examination object in the first state,
(230) feeding the at least one input image to the trained machine-learning model,
(240) receiving a synthetic image from the trained machine-learning model, where the synthetic image represents the examination region of the examination object in the second state,
(250) receiving an uncertainty value for each image element of the synthetic image,
(260) determining at least one confidence value on the basis of the received uncertainty values,
(270) outputting the at least one confidence value.

FIG. 6 shows in schematic form a further embodiment of the computer-implemented method for generating a synthetic image of an examination region of an examination object.

The synthetic image is generated using a trained machine-learning model MLM^{t}. The machine-learning model MLM^{t} can have been trained as described in relation to FIG. 4.

The trained machine-learning model MLM^{t} is fed at least one input image of an examination region of an examination object. The at least one input image represents the examination region of the examination object in a first state.

In the example shown in FIG. 6, the at least one input image comprises a first input image I₁(x₁ᵢ) and a second input image I₂(x₂ᵢ). The first input image I₁(x₁ᵢ) represents the examination region of the examination object in a first state and the second input image I₂(x₂ᵢ) represents the examination region of the examination object in the first state or in a second state.

For example, the first input image I₁(x₁ᵢ) can represent the examination region without administration or after administration of a first amount of a contrast agent and the second input image I₂(x₂ᵢ) can represent the examination region after administration of a second amount of the contrast agent. The first amount can be less than the second amount (see for example WO 2019/074938 A1, WO 2022/184297 A1).

For example, the first input image I₁(x₁ᵢ) can represent the examination region before administration or in a first period of time after administration of a contrast agent and the second input image I₂(x₂ᵢ) can represent the examination region in a second period of time after administration of the contrast agent. The second period of time can follow the first period of time (see for example WO 2021/052896 A1, WO 2021/069338 A1).

Further examples are mentioned in this disclosure.

The first input image I₁(x₁ᵢ) comprises a multiplicity of image elements (not explicitly shown in FIG. 6); each image element i of the first input image I₁(x₁ᵢ) represents a sub-region of the examination region; each image element i of the first input image I₁(x₁ᵢ) is assigned a color value xii.

The second input image I₂(x₂ᵢ) likewise comprises a multiplicity of image elements (not explicitly shown in FIG. 6); each image element i of the second input image I₂(x₂ᵢ) represents a sub-region of the examination region; each image element i of the second input image I₂(x₂ᵢ) is assigned a color value x₂ᵢ.

The trained machine-learning model MLM^{t} is configured and has been trained to generate on the basis of the first input image I₁(x₁ᵢ) and the second input image I₂(x₂ᵢ) a synthetic image I₃*(ŷᵢ).

The synthetic image I₃*(ŷᵢ) represents the examination region of the examination object in a third state. For example, the synthetic image I₃*(ŷᵢ) can represent the examination region after administration of a third amount of the contrast agent. The third amount can be greater than the second amount (see for example WO 2019/074938 A1, WO 2022/184297 A1). For example, the synthetic image I₃*(ŷᵢ) can represent the examination region in a third period of time after administration of the contrast agent. The third period of time can follow the second period of time (see for example WO 2021/052896 A1, WO 2021/069338 A1). Further examples are mentioned in this disclosure.

The synthetic image I₃*(ŷᵢ) can be output (i.e. for example displayed on a monitor or printed by a printer) and/or stored in a data memory and/or transmitted to a separate computer system.

The synthetic image I₃*(ŷᵢ) comprises a multiplicity of image elements (not explicitly shown in FIG. 6); each image element i of the synthetic image I₃*(ŷᵢ) represents a sub-region of the examination region.

The trained machine-learning model MLM^{t} is configured and has been trained to predict on the basis of the first input image I₁(x₁ᵢ) and the second input image I₂(x₂ᵢ) a color value ŷᵢ for each image element i of the synthetic image I₃*(ŷᵢ). In other words, each image element i of the synthetic image I₃*(ŷᵢ) is assigned a predicted color value ŷᵢ.

The trained machine-learning model MLM^{t} is furthermore configured and has furthermore been trained to predict for each predicted color value ŷᵢ an uncertainty value \hat{σ}(x₁ᵢ, x₂ᵢ).

The uncertainty values predicted for the predicted color values of the image elements of a synthetic image can be used as the basis to determine at least one confidence value.

The method for generating at least one confidence value for a synthetic image that is shown in FIG. 6 therefore comprises the following steps:
providing a trained machine-learning model,
   where the trained machine-learning model has been trained on the basis of training data,
   where the training data comprise for each reference object of a multiplicity of reference objects (i) a first input reference image of a reference region of the reference object in a first state and a second input reference image of the reference region of the reference object in a second state and (ii) a target reference image of the reference region of the reference object in a third state, where the first input reference image, the second input reference image and the target reference image each comprise a multiplicity of image elements,
   where the machine-learning model is configured and has been trained to generate for each reference object on the basis of the first input reference image and the second input reference image a synthetic reference image,
      where the synthetic reference image comprises a multiplicity of image elements, where each image element of the synthetic reference image respectively corresponds to an image element of the target reference image,
      where the machine-learning model has been trained to predict for each image element of the synthetic reference image a color value and an uncertainty value for the predicted color value,
      where the training comprises minimization of a loss function, where the loss function comprises (i) the predicted color value and/or a deviation of the predicted color value from a color value of the corresponding image element of the target reference image and (ii) the predicted uncertainty value as parameters,
receiving a first input image and a second input image of an examination region of an examination object, where the first input image represents the examination region of the examination object in the first state and the second input image represents the examination region of the examination object in the second state,
feeding the first input image and the second input image to the trained machine-learning model,
receiving a synthetic image from the trained machine-learning model, where the synthetic image represents the examination region of the examination object in the third state,
receiving an uncertainty value for each image element of the synthetic image,
determining at least one confidence value on the basis of the received uncertainty values,
outputting the at least one confidence value.

FIG. 7 shows by way of example and in schematic form a computer system according to the present disclosure.

A “computer system” is an electronic data processing system that processes data by means of programmable calculation rules. Such a system typically comprises a “computer”, which is the unit that includes a processor for carrying out logic operations, and peripherals.

In computer technology, “peripherals” denotes all devices that are connected to the computer and are used to control the computer and/or as input and output devices. Examples thereof are monitor (screen), printer, scanner, mouse, keyboard, drives, camera, microphone, speakers, etc. Internal ports and expansion cards are also regarded as peripherals in computer technology.

The computer system (10) shown in FIG. 7 comprises a receiving unit (11), a control and calculation unit (12) and an output unit (13).

The control and calculation unit (12) serves for control of the computer system (10), for coordination of the data flows between the units of the computer system (10), and for the performance of calculations.

The control and calculation unit (12) is configured:
to provide a trained machine-learning model,
   where the trained machine-learning model has been trained on the basis of training data,
   where the training data comprise for each reference object of a multiplicity of reference objects (i) at least one input reference image of a reference region of the reference object in a first state and (ii) a target reference image of the reference region of the reference object in a second state, where the at least one input reference image and the target reference image each comprise a multiplicity of image elements,
   where the machine-learning model is configured and has been trained to generate for each reference object on the basis of the at least one input reference image a synthetic reference image,
      where the synthetic reference image comprises a multiplicity of image elements, where each image element of the synthetic reference image respectively corresponds to an image element of the target reference image,
      where the machine-learning model has been trained to predict for each image element of the synthetic reference image a color value and an uncertainty value for the predicted color value,
      where the training comprises minimization of a loss function, where the loss function comprises (i) the predicted color value and/or a deviation of the predicted color value from a color value of the corresponding image element of the target reference image and (ii) the predicted uncertainty value as parameters,
to cause the receiving unit (11) to receive at least one input image of an examination region of an examination object, where the at least one input image represents the examination region of the examination object in the first state,
to feed the at least one input image to the trained machine-learning model,
to receive from the trained machine-learning model a synthetic image, wherein the synthetic image represents the examination region of the examination object in the second state,
to receive from the trained machine-learning model an uncertainty value for each image element of the synthetic image,
to determine at least one confidence value on the basis of the received uncertainty values,
to cause the output unit (13) to output the at least one confidence value, to store it in a data memory and/or to transmit it to a separate computer system.

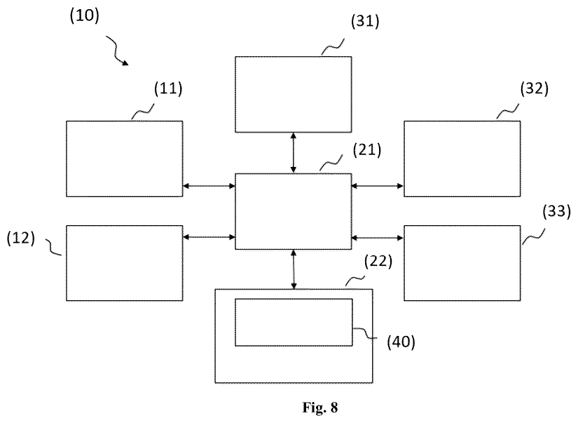

FIG. 8 shows by way of example and in schematic form a further embodiment of the computer system. The computer system (10) comprises a processing unit (21) connected to a memory (22). The processing unit (21) and the memory (22) form a control and calculation unit, as shown in FIG. 7.

The processing unit (21) may comprise one or more processors alone or in combination with one or more memories. The processing unit (21) may be customary computer hardware that is able to process information such as digital images, computer programs and/or other digital information. The processing unit (21) normally consists of an arrangement of electronic circuits, some of which can be designed as an integrated circuit or as a plurality of integrated circuits connected to one another (an integrated circuit is sometimes also referred to as a “chip”). The processing unit (21) may be configured to execute computer programs that can be stored in a working memory of the processing unit (21) or in the memory (22) of the same or of a different computer system.

The memory (22) may be customary computer hardware that is able to store information such as digital images (for example representations of the examination region), data, computer programs and/or other digital information either temporarily and/or permanently. The memory (22) may comprise a volatile and/or non-volatile memory and may be fixed in place or removable. Examples of suitable memories are RAM (random access memory), ROM (read-only memory), a hard disk, a flash memory, an exchangeable computer floppy disk, an optical disc, a magnetic tape or a combination of the aforementioned. Optical discs can include compact discs with read-only memory (CD-ROM), compact discs with read/write function (CD-R/W), DVDs, Blu-ray discs and the like.

The processing unit (21) may be connected not just to the memory (22), but also to one or more interfaces (11, 12, 31, 32, 33) in order to display, transmit and/or receive information. The interfaces may comprise one or more communication interfaces (11, 32, 33) and/or one or more user interfaces (12, 31). The one or more communication interfaces may be configured to send and/or receive information, for example to and/or from an MRI scanner, a CT scanner, an ultrasound camera, other computer systems, networks, data memories or the like. The one or more communication interfaces may be configured to transmit and/or receive information via physical (wired) and/or wireless communication connections. The one or more communication interfaces may comprise one or more interfaces for connection to a network, for example using technologies such as mobile telephone, wifi, satellite, cable, DSL, optical fibre and/or the like. In some examples, the one or more communication interfaces may comprise one or more close-range communication interfaces configured to connect devices having close-range communication technologies such as NFC, RFID, Bluetooth, Bluetooth LE, ZigBee, infrared (e.g. IrDA) or the like.

The user interfaces may include a display (31). A display (31) may be configured to display information to a user. Suitable examples thereof are a liquid crystal display (LCD), a light-emitting diode display (LED), a plasma display panel (PDP) or the like. The user input interface(s) (11, 12) may be wired or wireless and may be configured to receive information from a user in the computer system (1), for example for processing, storage and/or display. Suitable examples of user input interfaces are a microphone, an image- or video-recording device (for example a camera), a keyboard or a keypad, a joystick, a touch-sensitive surface (separate from a touchscreen or integrated therein) or the like. In some examples, the user interfaces may contain an automatic identification and data capture technology (AIDC) for machine-readable information. This can include barcodes, radiofrequency identification (RFID), magnetic strips, optical character recognition (OCR), integrated circuit cards (ICC) and the like. The user interfaces may in addition comprise one or more interfaces for communication with peripherals such as printers and the like.

One or more computer programs (40) may be stored in the memory (22) and executed by the processing unit (21), which is thereby programmed to fulfil the functions described in this description. The retrieving, loading and execution of instructions of the computer program (40) may take place sequentially, such that an instruction is respectively retrieved, loaded and executed. However, the retrieving, loading and/or execution may also take place in parallel.

The computer system of the present disclosure may be designed as a laptop, notebook, netbook and/or tablet PC; it may also be a component of an MRI scanner, a CT scanner or an ultrasound diagnostic device.

The present disclosure also provides a computer program product. Such a computer program product includes a non-volatile data carrier, for example a CD, a DVD, a USB stick or another data storage medium. Stored on the data carrier is a computer program. The computer program can be loaded into a working memory of a computer system (more particularly into a working memory of a computer system of the present disclosure), where the computer program causes the computer system to:
provide a trained machine-learning model,
   where the trained machine-learning model has been trained on the basis of training data,
   where the training data comprise for each reference object of a multiplicity of reference objects (i) at least one input reference image of a reference region of the reference object in a first state and (ii) a target reference image of the reference region of the reference object in a second state, where the at least one input reference image and the target reference image each comprise a multiplicity of image elements,
   where the machine-learning model is configured and has been trained to generate for each reference object on the basis of the at least one input reference image a synthetic reference image,
      where the synthetic reference image comprises a multiplicity of image elements, where each image element of the synthetic reference image respectively corresponds to an image element of the target reference image,
      where the machine-learning model has been trained to predict for each image element of the synthetic reference image a color value and an uncertainty value for the predicted color value,
      where the training comprises minimization of a loss function, where the loss function comprises (i) the predicted color value and/or a deviation of the predicted color value from a color value of the corresponding image element of the target reference image and (ii) the predicted uncertainty value as parameters,
receive at least one input image of an examination region of an examination object, where the at least one input image represents the examination region of the examination object in the first state,
feed the at least one input image to the trained machine-learning model,
receiving a synthetic image from the trained machine-learning model, where the synthetic image represents the examination region of the examination object in the second state,
receive an uncertainty value for each image element of the synthetic image,
determine at least one confidence value on the basis of the received uncertainty values,
output the at least one confidence value.

The computer program product can also be marketed in combination (in a set) with the contrast agent. Such a set is also referred to as a kit. Such a kit comprises the contrast agent and the computer program product. It is also possible for such a kit to comprise the contrast agent and means that allow the purchaser to obtain the computer program, for example by downloading it from a webpage. These means may include a link, i.e. an address of the webpage on which the computer program can be obtained, for example from which the computer program can be downloaded to a computer system connected to the internet. These means may include a code (for example an alphanumeric string or a QR code, or a DataMatrix code or a barcode or another optically and/or electronically readable code) that gives the purchaser access to the computer program. Such a link and/or code may for example be printed on a packaging of the contrast agent and/or on a package leaflet of the contrast agent. A kit is thus a combination product comprising a contrast agent and a computer program (for example in the form of access to the computer program or in the form of executable program code on a data carrier) that are offered for sale together.

1. A computer-implemented method comprising:
providing a trained machine-learning model (MLM^{t});
wherein the trained machine-learning model (MLM^{t}) has been trained on the basis of training data (TD),
wherein the training data (TD) comprise for each reference object of a plurality of reference objects (i) at least one input reference image (RI₁(xᵢ)) of a reference region of the reference object in a first state and (ii) a target reference image (RI₂(yᵢ)) of the reference region of the reference object in a second state, wherein the at least one input reference image (RI₁(xᵢ)) and the target reference image (RI₂(yᵢ)) each comprise a plurality of image elements, wherein the at least one input reference image (RI₁(xᵢ)) comprises at least one computed tomography or magnetic resonance image of the reference region of the reference object, wherein the target reference image (RI₂(yᵢ)) is a computed tomography or magnetic resonance image of the reference region of the reference object,
wherein the machine-learning model (MLM^{t}) is configured and has been trained to generate for each reference object on the basis of the at least one input reference image (RI₁(xᵢ)) a synthetic reference image (RI₂*(ŷᵢ)),
wherein the synthetic reference image (RI₂*(ŷᵢ)) comprises a plurality of image elements, wherein each image element of the synthetic reference image (RI₂*(ŷᵢ) respectively corresponds to an image element of the target reference image (RI₂(yᵢ)),
wherein the machine-learning model (MLM^{t}) has been trained to predict for each image element of the synthetic reference image (RI₂*(ŷᵢ)) a color value (ŷᵢ) and an uncertainty value (\hat{σ}(xᵢ)) for the predicted color value (ŷᵢ), and
wherein the training comprises minimization of a loss function (L), wherein the loss function (L) comprises (i) the predicted color value (ŷᵢ) or a deviation of the predicted color value (ŷᵢ) from a color value (yᵢ) of the corresponding image element of the target reference image (RI₂(yᵢ)) and (ii) the predicted uncertainty value (σ(xᵢ)) as parameters;
receiving at least one input image (I₁(xᵢ)) of an examination region of an examination object, wherein the at least one input image (I₁(xᵢ)) represents the examination region of the examination object in the first state, wherein the at least one input image (I₁(xᵢ)) comprises at least one computed tomography or magnetic resonance image of the examination region of the examination object;
feeding the at least one input image (I₁(xᵢ)) to the trained machine-learning model (MLM^{t});
receiving a synthetic image (I₂*(ŷᵢ)) from the trained machine-learning model, wherein the synthetic image (I₂*(ŷᵤ)) represents the examination region of the examination object in the second state;
receiving an uncertainty value (\hat{σ}(xᵢ)) for each image element of the synthetic image (I₂*(ŷᵢ));
determining at least one confidence value on the basis of the received uncertainty values; and
outputting the at least one confidence value.

2. The method according to claim 1, wherein the first state is a state before or after administration of a contrast agent and the second state is a state after administration of the contrast agent.

3. The method according to claim 1, wherein the first state and the second state each indicate an amount of a contrast agent which has been administered to the reference object and the examination object.

4. The method according to claim 1,
wherein the at least one input image (I₁(xᵢ)) comprises a first computed tomography or magnetic resonance image and a second computed tomography or magnetic resonance image, wherein the first computed tomography or magnetic resonance image represents the examination region of the examination object without a contrast agent or after administration of a first amount of the contrast agent and the second computed tomography or magnetic resonance image represents the examination region of the examination object after administration of a second amount of the contrast agent, and
wherein the synthetic image (I₂*(ŷᵢ)) is a synthetic computed tomography or magnetic resonance image, wherein the synthetic image (I₂*(ŷᵢ)) represents the examination region of the examination object after administration of a third amount of the contrast agent, wherein the second amount is different from, the first amount and the third amount is different from, the first amount and the second amount.

5. The method according to claim 1,
wherein the at least one input image (I₁(xᵢ)) comprises a first computed tomography or magnetic resonance image and a second computed tomography or magnetic resonance image, wherein the first computed tomography or magnetic resonance image represents the examination region of the examination object in a first period of time before or after administration of a contrast agent and the second computed tomography or magnetic resonance image represents the examination region of the examination object in a second period of time after administration of the contrast agent, and
wherein the synthetic image (I₂*(ŷᵢ)) is a synthetic computed tomography or magnetic resonance image, wherein the synthetic image (I₂*(ŷᵢ)) represents the examination region of the examination object in a third period of time after administration of the contrast agent, wherein the second period of time follows the first period of time, and the third period of time follows the second period of time.

6. The method according to claim 1, wherein the uncertainty value (\hat{σ}(xᵢ)) of the predicted color value (ŷᵢ) of each image element of the synthetic image (I₂*(ŷᵢ)) or a value derived therefrom is set as the confidence value of the image element.

7. The method according to claim 1, further comprising:
generating a confidence representation, wherein the confidence representation comprises a plurality of image elements, wherein each image element of the plurality of image elements represents a sub-region of the examination region, wherein each image element of the plurality of image elements respectively corresponds to an image element of the synthetic image (I₂*(ŷᵢ)), wherein each image element of the plurality of image elements has a color value, wherein the color value correlates with the respective uncertainty value (\hat{σ}(xᵢ)) of the predicted color value (ŷᵢ) of the corresponding image element of the synthetic image (I₂*(ŷᵢ)); and
outputting the confidence representation superimposed on the synthetic image (I₂*(ŷᵢ)).

8. The method according to claim 1, wherein the at least one confidence value is a confidence value for the entire synthetic image (I₂*(ŷ₁)), wherein the confidence value is a mean or a maximum value or a minimum value that is formed on the basis of all uncertainty values (\hat{σ}(xᵢ)) of all image elements of the synthetic image (I₂*(ŷᵢ)).

9. The method according to claim 1, further comprising:
determining a confidence value for one or more sub-regions of the synthetic image (I₂*(ŷᵢ)); and
outputting the confidence value for the one or more sub-regions of the synthetic image (I₂*(ŷᵢ)).

10. The method according to claim 9, wherein different methods for calculating the confidence value are used for different sub-regions of the examination region of the examination object.

11. The method according to claim 1, wherein the trained machine-learning model (MLM^{t}) is configured and has been trained to increase, in the event of an increase in the deviation of the predicted color value (ŷᵢ) of the synthetic reference image (RI₂*(ŷᵢ)) from the color value (yᵢ) of the corresponding image element of the target reference image (RI₂(yᵢ)), the uncertainty value (\hat{σ}(xᵢ)) of the predicted color value (ŷᵢ) in order to minimize the loss function.

12. The method according to claim 1, wherein an increase in the deviation of the predicted color value (ŷᵢ) of the synthetic reference image (RI₂*(ŷᵢ)) from the color value (yᵢ) of the corresponding image element of the target reference image (RI₂(yᵢ)) leads to an increase in a loss calculated by means of the loss function ( [symbol] ), wherein an increase in the uncertainty value (\hat{σ}(xᵢ)) leads to a decrease in the loss calculated by means of the loss function ( [symbol] ).

13. The method according to claim 1, wherein the loss function ( [symbol] ) comprises the following equation (1):
$$ℒ = \frac{1}{N} {\sum }_{i = 1}^{N} \frac{1}{2 {\hat{σ} ( {x}_{i} )}^{2}} ‖ {y}_{i} - {\hat{y}}_{i} {‖}^{2} + \frac{1}{2} log {\hat{σ} ( {x}_{i} )}^{2} \qquad ( 1 )$$
wherein N is the number of image elements of the synthetic reference image (RI₂*(ŷᵢ)), ŷᵢ is the predicted color value of the image element i of the synthetic reference image (RI₂*(ŷᵢ)), \hat{σ}(xᵢ) is the uncertainty value of the predicted color value of the image element i, and yᵢ is the color value of the corresponding image element of the target reference image (RI₂(yᵢ)).

14. The method according to claim 1, wherein the training of a machine-learning model (MLM) comprises:
receiving the training data (TD);
wherein the training data (TD) comprise for each reference object of the plurality of reference objects (i) the at least one input reference image (RI₁(xᵢ)) of the reference region of the reference object in the first state and (ii) the target reference image (RI₂(yᵢ)) of the reference region of the reference object in the second state,
wherein the second state is different from the first state,
wherein the at least one input reference image (RI₁(xᵢ)) comprises a plurality of image elements, wherein each image element of the at least one input reference image (RI₁(xᵢ)) represents a sub-region of the reference region, wherein each image element of the at least one input reference image (RI₁(xᵢ)) is characterized by a color value (xᵢ), and
wherein the target reference image (RI₂(yᵢ)) comprises a plurality of image elements, wherein each image element of the target reference image (RI₂(yᵢ)) represents a sub-region of the reference region, wherein each image element of the target reference image (RI₂(yᵢ)) is characterized by a color value (yᵢ);
providing the machine-learning model (MLM);
wherein the machine-learning model (MLM) is configured to generate on the basis of the at least one input reference image (RI₁(xᵢ)) of the reference region of a reference object and model parameters (MP) of a synthetic reference image (RI₂*(ŷᵢ)) of the reference region of the reference object, wherein the synthetic reference image (RI₂*(ŷᵢ)) comprises a plurality of image elements, wherein each image element of the synthetic reference image (RI₂*(ŷᵢ)) corresponds to an image element of the target reference image (RI₂(yᵢ)), wherein each image element of the synthetic reference image (RI₂*(ŷᵢ)) is assigned a predicted color value (ŷᵢ), wherein the machine-learning model (MLM) is configured to predict for each predicted color value (yî) an uncertainty value (\hat{σ}(xᵢ));
training the machine-learning model (MLM), wherein the training for each reference object of the plurality of reference objects comprises:
inputting the at least one input reference image (RI₁(xᵢ)) into the machine-learning model (MLM);
receiving the synthetic reference image (RI₂*(ŷᵢ)) from the machine-learning model (MLM);
receiving an uncertainty value (\hat{σ}(xᵢ)) for each predicted color value (ŷᵢ) of the synthetic reference image (RI₂*(ŷᵢ));
calculating a loss by means of a loss function ( [symbol] ), wherein the loss function ( [symbol] ) comprises (i) the predicted color value (ŷᵢ) or a deviation between the predicted color value (ŷᵢ) and a color value (yᵢ) of the corresponding image element of the target reference image (RI₂(yᵢ)) and (ii) the predicted uncertainty value (\hat{σ}(xᵢ)) as parameters; and
reducing the loss by modification of model parameters (MP); and
outputting and storing the trained machine-learning model (MLM^{t}) or transmitting the trained machine-learning model (MLM^{t}) to a separate computer system; and
using the trained machine-learning model (MLM^{t}) to predict a synthetic image and to generate at least one confidence value for a synthetic image.

15. A computer system comprising:
a receiving unit;
a control and calculation unit; and
an output unit;
wherein the control and calculation unit is configured to:
provide a trained machine-learning model (MLM^{t});
wherein the trained machine-learning model (MLM^{t}) has been trained on the basis of training data (TD),
wherein the training data (TD) comprise for each reference object of a plurality of reference objects (i) at least one input reference image (RI₁(xᵢ)) of a reference region of the reference object in a first state and (ii) a target reference image (RI₂(yᵢ)) of the reference region of the reference object in a second state, wherein the at least one input reference image (RI₁(xᵢ)) and the target reference image (RI₂(yᵢ)) each comprise a plurality of image elements, wherein the at least one input reference image (RI₁(xᵢ)) comprises at least one computed tomography or magnetic resonance image of the reference region of the reference object, wherein the target reference image (RI₂(yᵢ)) is a computed tomography or magnetic resonance image of the reference region of the reference object,
wherein the machine-learning model (MLM^{t}) is configured and has been trained to generate for each reference object on the basis of the at least one input reference image (RI₁(xᵢ)) a synthetic reference image (RI₂*(ŷᵢ)),
wherein the synthetic reference image (RI₂*(ŷᵢ)) comprises a plurality of image elements, wherein each image element of the synthetic reference image (RI₂*(ŷᵢ)) respectively corresponds to an image element of the target reference image (RI₂(yᵢ)),
wherein the machine-learning model (MLM^{t}) has been trained to predict for each image element of the synthetic reference image (RI₂*(ŷᵢ)) a color value (ŷᵢ) and an uncertainty value (\hat{σ}(xᵢ)) for the predicted color value (ŷᵢ), and
wherein the training comprises minimization of a loss function ( [symbol] ), wherein the loss function ( [symbol] ) comprises (i) the predicted color value (ŷᵢ) or a deviation of the predicted color value (ŷᵢ) from a color value (yᵢ) of the corresponding image element of the target reference image (RI₂(yᵢ)) and (ii) the predicted uncertainty value (\hat{σ}(xᵢ)) as parameters;
cause the receiving unit to receive at least one input image (I₁(xᵢ)) of an examination region of an examination object, wherein the at least one input image (I₁(xᵢ)) represents an examination region of an examination object in the first state, wherein the at least one input image (I₁(xᵢ)) comprises at least one computed tomography or magnetic resonance image of the examination region of the examination object;
feed the at least one input image (I₁(xᵢ)) to a trained machine-learning model (MLM^{t});
receive from the trained machine-learning model (MLM^{t}) a synthetic image (I₂*(ŷᵢ)), wherein the synthetic image (I₂*(ŷᵢ)) represents the examination region of the examination object in the second state;
receive from the trained machine-learning model (MLM^{t}) an uncertainty value (\hat{σ}(xᵢ)) for each image element of the synthetic image (I₂*(ŷᵢ));
determine at least one confidence value on the basis of the received uncertainty values; and
cause the output unit to output the at least one confidence value, store it in a data memory and transmit it to a separate computer system.

16. A computer-readable storage medium comprising a computer program which, when loaded into a working memory of a computer system, causes the computer system to execute:
providing a trained machine-learning model (MLM^{t});
wherein the trained machine-learning model (MLM^{t}) has been trained on the basis of training data (TD),
wherein the training data (TD) comprise for each reference object of a plurality of reference objects (i) at least one input reference image (RI₁(xᵢ)) of a reference region of the reference object in a first state and (ii) a target reference image (RI₂(yᵢ)) of the reference region of the reference object in a second state, wherein the at least one input reference image (RI₁(xᵢ)) and the target reference image (RI₂(yᵢ)) each comprise a plurality of image elements, wherein the at least one input reference image (RI₁(xᵢ)) comprises at least one computed tomography or magnetic resonance image of the reference region of the reference object, wherein the target reference image (RI₂(yᵢ)) is a computed tomography or magnetic resonance image of the reference region of the reference object,
wherein the machine-learning model (MLM^{t}) is configured and has been trained to generate for each reference object on the basis of the at least one input reference image (RI₁(xᵢ)) a synthetic reference image (RI₂*(ŷᵢ)),
wherein the synthetic reference image (RI₂*(ŷᵢ)) comprises a plurality of image elements, wherein each image element of the synthetic reference image (RI₂*(ŷᵢ)) respectively corresponds to an image element of the target reference image (RI₂(yᵢ)),
wherein the machine-learning model (MLM^{t}) has been trained to predict for each image element of the synthetic reference image (RI₂*(ŷᵢ)) a color value (ŷᵢ) and an uncertainty value (\hat{σ}(xᵢ)) for the predicted color value (ŷᵢ), and
wherein the training comprises minimization of a loss function ( [symbol] ), wherein the loss function ( [symbol] ) comprises (i) the predicted color value (ŷᵢ) or a deviation of the predicted color value (ŷᵢ) from a color value (yᵢ) of the corresponding image element of the target reference image (RI₂(yᵢ)) and (ii) the predicted uncertainty value (\hat{σ}(xᵢ)) as parameters;
receiving at least one input image (I₁(xᵢ)) of an examination region of an examination object, wherein the at least one input image (I₁(xᵢ)) represents the examination region of the examination object in the first state, wherein the at least one input image (I₁(xᵢ)) comprises at least one computed tomography or magnetic resonance image of the examination region of the examination object;
feeding the at least one input image (I₁(xᵢ)) to the trained machine-learning model (MLM^{t});
receiving a synthetic image (I₂*(ŷᵢ)) from the trained machine-learning model, wherein the synthetic image (I₂*(ŷᵢ)) represents the examination region of the examination object in the second state;
receiving an uncertainty value (\hat{σ}(xᵢ)) for each image element of the synthetic image (I₂*(ŷᵢ));
determining at least one confidence value on the basis of the received uncertainty values; and
outputting the at least one confidence value.

17. A contrast agent for use in a radiological examination method, the method comprising:
providing a trained machine-learning model (MLM^{t});
wherein the trained machine-learning model (MLM^{t}) has been trained on the basis of training data (TD),
wherein the training data (TD) comprise for each reference object of a plurality of reference objects (i) at least one input reference image (RI₁(xᵢ)) of a reference region of the reference object in a first state and (ii) a target reference image (RI₂(yᵢ)) of the reference region of the reference object in a second state,
wherein the at least one input reference image (RI₁(xᵢ)) and the target reference image (RI₂(yᵢ)) each comprise a plurality of image elements,
wherein the at least one input reference image (RI₁(xᵢ)) comprises at least one computed tomography or magnetic resonance image of the reference region of the reference object in the first state,
wherein the target reference image (RI₂(yᵢ)) is a computed tomography or magnetic resonance image of the reference region of the reference object in the second state, and
wherein the first state represents the reference region of the reference object in a first period of time before or after the administration of the contrast agent and the second state represents the reference region of the reference object in a second period of time after the administration of the contrast agent, and/or the first state represents the reference region of the reference object before or after the administration of a first amount of the contrast agent and the second state represents the reference region of the reference object after the administration of a second amount of the contrast agent,
wherein the machine-learning model (MLM^{t}) is configured and has been trained to generate for each reference object on the basis of the at least one input reference image (RI₁(xᵢ)) a synthetic reference image (RI₂*(ŷᵢ)),
wherein the synthetic reference image (RI₂*(ŷᵢ)) comprises a plurality of image elements, wherein each image element of the synthetic reference image (RI₂*(ŷᵢ)) respectively corresponds to an image element of the target reference image (RI₂(yᵢ)),
wherein the machine-learning model (MLM^{t}) has been trained to predict for each image element of the synthetic reference image (RI₂*(ŷᵢ)) a color value (ŷᵢ) and an uncertainty value (\hat{σ}(xᵢ)) for the predicted color value (ŷᵢ), and
wherein the training comprises minimization of a loss function ( [symbol] ), wherein the loss function ( [symbol] ) comprises (i) the predicted color value (ŷᵢ) or a deviation of the predicted color value (ŷᵢ) from a color value (yᵢ) of the corresponding image element of the target reference image (RI₂(yᵢ)) and (ii) the predicted uncertainty value (\hat{σ}(xᵢ)) as parameters;
receiving at least one input image (I₁(xᵢ)) of an examination region of an examination object, wherein the at least one input image (I₁(xᵢ)) comprises at least one computed tomography or magnetic resonance image of the examination region of the examination object in the first state;
feeding the at least one input image (I₁(xᵢ)) to the trained machine-learning model (MLM^{t});
receiving a synthetic image (I₂*(ŷᵢ)) from the trained machine-learning model, wherein the synthetic image (I₂*(ŷᵢ)) comprises a synthetic radiological image representing the examination region of the examination object in the second state;
receiving an uncertainty value (\hat{σ}(xᵢ)) for each image element of the synthetic image (I₂*(ŷᵢ));
determining at least one confidence value on the basis of the received uncertainty values; and
outputting the at least one confidence value.

18. The method according to claim 2, wherein the contrast agent comprises one or more of the following compounds:
gadolinium(III) 2-[4,7,10-tris(carboxymethyl)-1,4,7,10-tetrazacyclododec-1-yl]acetic acid;
gadolinium(III) ethoxybenzyldiethylenetriaminepentaacetic acid;
gadolinium(III) 2-[3,9-bis[1-carboxylato-4-(2,3-dihydroxypropylamino)-4-oxobutyl]-3,6,9,15-tetrazabicyclo[9.3.1]pentadeca-1(15),11,13-trien-6-yl]-5-(2,3-dihydroxypropylamino)-5-oxopentanoate;
dihydrogen [(±)-4-carboxy-5,8,11-tris(carboxymethyl)-1-phenyl-2-oxa-5,8,11-triazatridecan-13-oato(5-)]gadolinate(2-);
tetragadolinium [4,10-bis(carboxylatomethyl)-7-{3,6,12,15-tetraoxo-16-[4,7,10-tris(carboxylatomethyl)-1,4,7,10-tetraazacyclododecan-1-yl]-9,9-bis({[({2-[4,7,10-tris(carboxylatomethyl)-1,4,7,10-tetraazacyclododecan-1-yl]propanoyl}amino)acetyl]amino}methyl)-4,7,11,14-tetraazaheptadecan-2-yl}-1,4,7,10-tetraazacyclododecan-1-yl]acetate;
gadolinium 2,2′,2″-(10-{1-carboxy-2-[2-(4-ethoxyphenyl)ethoxy]ethyl}-1,4,7,10-tetraazacyclododecane-1,4,7-triyl)triacetate;
gadolinium 2,2′,2″-{10-[1-carboxy-2-{4-[2-(2-ethoxyethoxy)ethoxy]phenyl}ethyl]-1,4,7,10-tetraazacyclododecane-1,4,7-triyl}triacetate;
gadolinium 2,2′,2″-{10-[(1R)-1-carboxy-2-{4-[2-(2-ethoxyethoxy)ethoxy]phenyl}ethyl]-1,4,7,10-tetraazacyclododecane-1,4,7-triyl}triacetate;
gadolinium (2S,2′S,2″S)-2,2′,2″-{10-[(1S)-1-carboxy-4-{4-[2-(2-ethoxyethoxy)ethoxy]phenyl}butyl]-1,4,7,10-tetraazacyclododecane-1,4,7-triyl}tris(3-hydroxypropanoate);
gadolinium 2,2′,2″-{10-[(1S)-4-(4-butoxyphenyl)-1-carboxybutyl]-1,4,7,10-tetraazacyclododecane-1,4,7-triyl}triacetate;
gadolinium 2,2′,2″-{(2S)-10-(carboxymethyl)-2-[4-(2-ethoxyethoxy)benzyl]-1,4,7,10-tetraazacyclododecane-1,4,7-triyl}triacetate;
gadolinium 2,2′,2″-[10-(carboxymethyl)-2-(4-ethoxybenzyl)-1,4,7,10-tetraazacyclododecane-1,4,7-triyl]triacetate;
gadolinium(III) 5,8-bis(carboxylatomethyl)-2-[2-(methylamino)-2-oxoethyl]-10-oxo-2,5,8,11-tetraazadodecane-1-carboxylate hydrate;
gadolinium(III) 2-[4-(2-hydroxypropyl)-7,10-bis(2-oxido-2-oxoethyl)-1,4,7,10-tetrazacyclododec-1-yl]acetate;
gadolinium(III) 2,2′,2″-(10-((2R,3S)-1,3,4-trihydroxybutan-2-yl)-1,4,7,10-tetraazacyclododecane-1,4,7-triyl)triacetate;
a Gd³⁺ complex of a compound of formula (I) 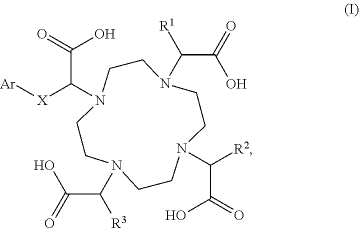
wherein:
Ar is a group selected from: 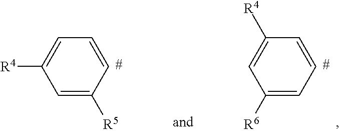
wherein _{#} is a linkage to X;
X is a group selected from:
CH₂, (CH₂)₂, (CH₂)₃, (CH₂)₄ and *—(CH₂)₂—O—CH₂—^{#};
wherein * is a linkage to Ar and ^{#} is a linkage to an acetic acid residue;
R¹, R² and R³ are each independently a hydrogen atom or a group selected from C₁-C₃ alkyl, —CH₂OH, —(CH₂)₂OH and —CH₂OCH₃;
R⁴ is a group selected from C₂-C₄ alkoxy, (H₃C—CH₂)—O—(CH₂)₂—O—, (H₃C—CH₂)—O—(CH₂)₂—O—(CH₂)₂—O— and (H₃C—CH₂)—O—(CH₂)₂—O—(CH₂)₂—O—(CH₂)₂—O—;
R⁵ is a hydrogen atom; and
R⁶ is a hydrogen atom;
or a stereoisomer, a tautomer, a hydrate, a solvate or a salt thereof, or a mixture thereof,
a Gd³⁺ complex of a compound of formula (II) 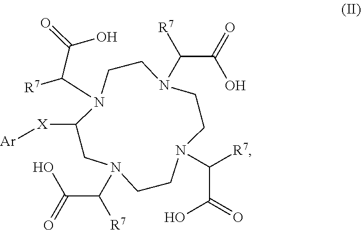
wherein:
Ar is a group selected from: 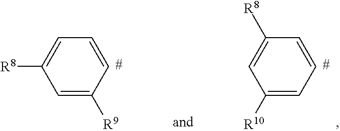
wherein _{#} is a linkage to X;
X is a group selected from:
CH₂, (CH₂)₂, (CH₂)₃, (CH₂)₄ and *—(CH₂)₂—O—CH₂—^{#};
wherein * is a linkage to Ar and ^{#} is a linkage to an acetic acid residue;
R⁷ is a hydrogen atom or a group selected from C₁-C₃ alkyl, —CH₂OH, —(CH₂)₂OH and —CH₂OCH₃;
R⁸ is a group selected from:
C₂-C₄ alkoxy, (H₃C—CH₂O)—(CH₂)₂—O—, (H₃C—CH₂O)—(CH₂)₂—O—(CH₂)₂—O— and (H₃C—CH₂O)—(CH₂)₂—O—(CH₂)₂—O—(CH₂)₂—O—;
R⁹ and R¹⁰ are independently a hydrogen atom;
or a stereoisomer, a tautomer, a hydrate, a solvate or a salt thereof, or a mixture thereof.

19. The method according to claim 17, wherein the contrast agent comprises one or more of the following compounds:
gadolinium(III) 2-[4,7,10-tris(carboxymethyl)-1,4,7,10-tetrazacyclododec-1-yl]acetic acid;
gadolinium(III) ethoxybenzyldiethylenetriaminepentaacetic acid;
gadolinium(III) 2-[3,9-bis[1-carboxylato-4-(2,3-dihydroxypropylamino)-4-oxobutyl]-3,6,9,15-tetrazabicyclo[9.3.1]pentadeca-1(15),11,13-trien-6-yl]-5-(2,3-dihydroxypropylamino)-5-oxopentanoate;
dihydrogen [(±)-4-carboxy-5,8,11-tris(carboxymethyl)-1-phenyl-2-oxa-5,8,11-triazatridecan-13-oato(5-)]gadolinate(2-);
tetragadolinium [4,10-bis(carboxylatomethyl)-7-{3,6,12,15-tetraoxo-16-[4,7,10-tris(carboxylatomethyl)-1,4,7,10-tetraazacyclododecan-1-yl]-9,9-bis({[({2-[4,7,10-tris(carboxylatomethyl)-1,4,7,10-tetraazacyclododecan-1-yl]propanoyl}amino)acetyl]amino}methyl)-4,7,11,14-tetraazaheptadecan-2-yl}-1,4,7,10-tetraazacyclododecan-1-yl]acetate;
gadolinium 2,2′,2″-(10-{1-carboxy-2-[2-(4-ethoxyphenyl)ethoxy]ethyl}-1,4,7,10-tetraazacyclododecane-1,4,7-triyl)triacetate;
gadolinium 2,2′,2″-{10-[1-carboxy-2-{4-[2-(2-ethoxyethoxy)ethoxy]phenyl}ethyl]-1,4,7,10-tetraazacyclododecane-1,4,7-triyl}triacetate;
gadolinium 2,2′,2″-{10-[(1R)-1-carboxy-2-{4-[2-(2-ethoxyethoxy)ethoxy]phenyl}ethyl]-1,4,7,10-tetraazacyclododecane-1,4,7-triyl}triacetate;
gadolinium (2S,2′S,2″S)-2,2′,2″-{10-[(1S)-1-carboxy-4-{4-[2-(2-ethoxyethoxy)ethoxy]phenyl}butyl]-1,4,7,10-tetraazacyclododecane-1,4,7-triyl}tris(3-hydroxypropanoate);
gadolinium 2,2′,2″-{10-[(1S)-4-(4-butoxyphenyl)-1-carboxybutyl]-1,4,7,10-tetraazacyclododecane-1,4,7-triyl}triacetate;
gadolinium 2,2′,2″-{(2S)-10-(carboxymethyl)-2-[4-(2-ethoxyethoxy)benzyl]-1,4,7,10-tetraazacyclododecane-1,4,7-triyl}triacetate;
gadolinium 2,2′,2″-[10-(carboxymethyl)-2-(4-ethoxybenzyl)-1,4,7,10-tetraazacyclododecane-1,4,7-triyl]triacetate;
gadolinium(III) 5,8-bis(carboxylatomethyl)-2-[2-(methylamino)-2-oxoethyl]-10-oxo-2,5,8,11-tetraazadodecane-1-carboxylate hydrate;
gadolinium(III) 2-[4-(2-hydroxypropyl)-7,10-bis(2-oxido-2-oxoethyl)-1,4,7,10-tetrazacyclododec-1-yl]acetate;
gadolinium(III) 2,2′,2″-(10-((2R,3S)-1,3,4-trihydroxybutan-2-yl)-1,4,7,10-tetraazacyclododecane-1,4,7-triyl)triacetate;
a Gd³⁺ complex of a compound of formula (I) 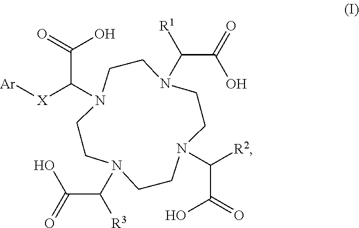
wherein:
Ar is a group selected from: 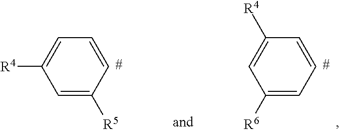
wherein _{#} is a linkage to X;
X is a group selected from:
CH₂, (CH₂)₂, (CH₂)₃, (CH₂)₄ and *—(CH₂)₂O—CH₂—^{#};
wherein * is a linkage to Ar and ^{#} is a linkage to an acetic acid residue;
R¹, R² and R³ are each independently a hydrogen atom or a group selected from C₁-C₃ alkyl, —CH₂OH, —(CH₂)₂OH and —CH₂OCH₃;
R⁴ is a group selected from C₂-C₄ alkoxy, (H₃C—CH₂)—O—(CH₂)₂—O—, (H₃C—CH₂)—O—(CH₂)₂—O—(CH₂)₂—O— and (H₃C—CH₂)—O—(CH₂)₂—O—(CH₂)₂—O—(CH₂)₂—O—;
R⁵ is a hydrogen atom; and
R⁶ is a hydrogen atom;
or a stereoisomer, a tautomer, a hydrate, a solvate or a salt thereof, or a mixture thereof,
a Gd³⁺ complex of a compound of formula (II) 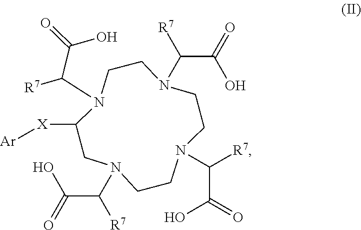
wherein:
Ar is a group selected from: 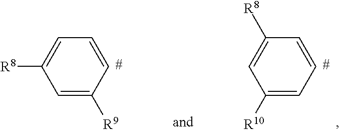
wherein _{#} is a linkage to X;
X is a group selected from:
CH₂, (CH₂)₂, (CH₂)₃, (CH₂)₄ and *—(CH₂)₂—O—CH₂—^{#};
wherein * is a linkage to Ar and ^{#} is a linkage to an acetic acid residue;
R⁷ is a hydrogen atom or a group selected from C₁-C₃ alkyl, —CH₂OH, —(CH₂)₂OH and —CH₂OCH₃;
R⁸ is a group selected from:
C₂-C₄ alkoxy, (H₃C—CH₂O)—(CH₂)₂—O—, (H₃C—CH₂O)—(CH₂)₂—O—(CH₂)₂—O— and (H₃C—CH₂O)—(CH₂)₂—O—(CH₂)₂—O—(CH₂)₂—O—;
R⁹ and R¹⁰ are independently a hydrogen atom;
or a stereoisomer, a tautomer, a hydrate, a solvate or a salt thereof, or a mixture thereof.

20. A kit comprising a contrast agent and a computer program which can be loaded into a working memory of a computer system, wherein the computer program causes the computer system to:
provide a trained machine-learning model (MLM^{t}),
wherein the trained machine-learning model (MLM^{t}) has been trained on the basis of training data (TD),
wherein the training data (TD) comprise for each reference object of a plurality of reference objects (i) at least one input reference image (RI₁(xᵢ)) of a reference region of the reference object in a first state and (ii) a target reference image (RI₂(yᵢ)) of the reference region of the reference object in a second state,
wherein the at least one input reference image (RI₁(xᵢ)) and the target reference image (RI₂(yᵢ)) each comprise a plurality of image elements,
wherein the at least one input reference image (RI₁(xᵢ)) comprises at least one computed tomography or magnetic resonance image of the reference region of the reference object in the first state,
wherein the target reference image (RI₂(yᵢ)) is a computed tomography or magnetic resonance image of the reference region of the reference object in the second state, and
wherein the first state represents the reference region of the reference object in a first period of time before or after the administration of the contrast agent and the second state represents the reference region of the reference object in a second period of time after the administration of the contrast agent, and/or the first state represents the reference region of the reference object before or after the administration of a first amount of the contrast agent and the second state represents the reference region of the reference object after the administration of a second amount of the contrast agent,
wherein the machine-learning model (MLM^{t}) is configured and has been trained to generate for each reference object on the basis of the at least one input reference image (RI₁(xᵢ)) a synthetic reference image (RI₂*(ŷᵢ)),
wherein the synthetic reference image (RI₂*(ŷᵢ)) comprises a plurality of image elements, wherein each image element of the synthetic reference image (RI₂*(ŷᵢ)) respectively corresponds to an image element of the target reference image (RI₂(yᵢ)),
wherein the machine-learning model (MLM^{t}) has been trained to predict for each image element of the synthetic reference image (RI₂*(ŷᵢ)) a color value (ŷᵢ) and an uncertainty value (\hat{σ}(xᵢ)) for the predicted color value (ŷᵢ), and
wherein the training comprises minimization of a loss function ( [symbol] ), wherein the loss function ( [symbol] ) comprises (i) the predicted color value (ŷᵢ) or a deviation of the predicted color value (ŷᵢ) from a color value (yᵢ) of the corresponding image element of the target reference image (RI₂(yᵢ)) and (ii) the predicted uncertainty value (\hat{σ}(xᵢ)) as parameters;
receive at least one input image (I₁(xᵢ)) of an examination region of an examination object, wherein the at least one input image (I₁(xᵢ)) comprises at least one computed tomography or magnetic resonance image of the examination region of the examination object in the first state;
feed the at least one input image (I₁(xᵢ)) to the trained machine-learning model (MLM^{t});
receive a synthetic image (I₂*(ŷᵢ)) from the trained machine-learning model, wherein the synthetic image (I₂*(ŷᵢ)) comprises a synthetic radiological image representing the examination region of the examination object in the second state;
receive an uncertainty value (\hat{σ}(xᵢ)) for each image element of the synthetic image (I₂*(ŷᵢ));
determine at least one confidence value on the basis of the received uncertainty values; and
output the at least one confidence value.
